(19) United States
(12) Patent Application Publication
(10) Pub. No.: US 2024/0398996 A1
(43) Pub. Date: Dec. 5, 2024

(54) COMPOSITIONS

(71) Applicant: Imperial College Innovations Ltd, London (GB)

(72) Inventors: Fang Xie, London (GB); Mary Ryan, London (GB); Caoimhe Joyce, London (GB); Tanveer Tabish, London (GB); Savita Sundaresan, London (GB); Jiamin Xu, London (GB)

(21) Appl. No.: 18/721,853

(22) PCT Filed: Dec. 19, 2022
(86) PCT No.: PCT/GB2022/053284

§ 371 Date: Jun. 19, 2024

(30) Foreign Application Priority Data
Dec. 20, 2021 (GB) 2118510.3

(51) Int. Cl. A61K 49/00

(52) U.S. Cl. CPC A61K 49/0034; A61K 49/0067; A61K 49/0095

(57) ABSTRACT

The present invention provides compositions and methods for enhancing fluorescence from emitters that emit in the NIR-II and NIR-III. The compositions have particular use in both in vitro diagnostics, and in the live imaging of tissue during surgery, for example during removal of a tumour.

### FIELD

The invention is in the field of visual aids to be employed in surgery.

### BACKGROUND

The second and third near infra-red (NIR-II and NIR-III) windows hold great promise for enhanced resolution in situ fluorescence imaging of biological tissue for preclinical and clinical applications. However, currently available NIR-II and NIR-III dyes display low quantum yields, thus limiting their biomedical applications.

During cancer surgery, it is vital to know what areas to resect while preserving healthy material that provide important functions, such as nervous, lymphatic and vasculature tissue. There is therefore an urgent need to improve real-time bioimaging techniques to aid surgeons in visualising different tissue types, particularly to distinguish cancerous and healthy material.

Fluorescence imaging can provide molecular contrast with high sensitivity and is a ubiquitous tool to study cell biology. In biological tissue, however, fluorescence signals can be swamped by autofluorescence background and image quality can be degraded by optical scattering. These issues can both be ameliorated by utilising near infrared (NIR) radiation since the molecular energy levels associated with most autofluorescence emission are excited by ultraviolet or visible radiation and the longer wavelength NIR radiation scatters less than visible radiation in biological tissue. The lower absorption of NIR radiation also results in reduced phototoxicity.

The NIR-II and NIR-III windows (1000-1400 nm; 1400-1700 nm) offer lower optical scattering, autofluorescence and phototoxicity than the NIR-I (650-900 nm) window with significantly less absorption from water and biological tissues such as haemoglobin, meaning that light is capable of penetrating deeper into soft tissue and blood¹. Furthermore, autofluorescence almost completely disappears at wavelengths above 1200 nm² and numerous studies have shown the superior tissue transparency and depth penetration that the NIR-II/NIR-III windows exhibits versus the NIR-I window³⁻⁵. Unfortunately, the exploitation of the NIR-II/NIR-III window has been held back by the lack of suitable light sources and detectors in this spectral region and the paucity of efficient NIR-II/NIR-III fluorophores. However, the situation is changing rapidly with the recent improved availability of laser-based sources and more cost-effective InGaAs cameras. It is therefore timely to address the unmet need for efficient NIR-II/NIR-III fluorophores.

Although several fluorescent NIR-II and NIR-III molecules are currently in development, none are FDA-approved and none have a quantum efficiency above 10%6. In fact, the widely used clinically approved NIR-I dye⁷, indocyanine green (ICG), has been shown to work as a NIR-II fluorophores, with brightness comparable to current specially designed NIR-II emitters. However, ICG still presents a quantum efficiency below 10% in the NIR-II window, meaning that a large amount of dye would be needed in order to produce a sufficiently bright signal for imaging in tissue.

There is therefore a need for strategies to increase the quantum efficiency of fluorescent NIR-II and NIR-III molecules so that a physiologically appropriate amount of dye may be used during, for example, surgery so as to enhance the ability of the surgeon to accurately remove cancerous tissue.

### SUMMARY OF THE INVENTION

The inventors surprisingly found that nanostructures of a particular composition have the ability to enhance fluorescence from emitters in the NIR-II and NIR-III windows. The enhanced fluorescence occurs through metal enhanced fluorescence (MEF). This is an optical process whereby, through the interaction between an emitter such as a fluorophore and a plasmonic nanoparticle (usually gold or silver) under specific conditions, an increase in the fluorescence intensity of the emitter, for example the fluorophore, can be observed⁹⁻¹¹. The magnitude of such enhancement is highly dependent on a number of factors, one of which being the spectral overlap between the localised surface plasmon resonance (LSPR) of the metal nanoparticle and the absorption and emission of the emitter^{12,13}.

### DETAILED DESCRIPTION

MEF is usually performed with solid metallic nanoparticles. However, the inventors of the present invention have surprisingly found that to enhance fluorescence in the NIR-II and NIR-III region of the spectrum, it is necessary to use nanoparticles that comprise a dielectric core, or a hollow core, with a metallic plasmonic coating that optionally comprises spikes of the metallic plasmonic material.

Accordingly, the invention provides a nanostructure, wherein the nanostructure comprises:
a) a dielectric core material and/or a hollow core;
b) a metallic plasmonic material; and
c) an emitter that absorbs and/or emits electromagnetic radiation in the near infra-red II window (NIR-II) and/or in the near infra-red III (NIR-III) window;
wherein the metallic plasmonic material covers substantially all, or covers all, of the dielectric core, and optionally forms at least one or more spikes on the surface of the core.

Nanostructure and nanoparticle are to be used interchangeably.

The nanostructure of the invention does not comprise a metal core, for example does not comprise a core containing or comprising cobalt.

The nanostructure of the invention does not comprise a magnetic core.

The nanostructures of the invention are considered to be useful in the context of light enhancement for Metal Enhanced Fluorescence (MEF) technology based biosensing/bioimaging applications. The nanostructures of the invention are considered to be useful in combining the functionality of imaging/biosensing with or without photothermal therapy. The nanostructures of the invention are not considered to be used in SERS spectroscopy or MRI imaging.

In preferred embodiments, the NIR-II window is defined as electromagnetic radiation of a wavelength of between 1,000 nm to 1,400 nm; and the NIR-III window is defined as electromagnetic radiation of a wavelength of 1400 nm to 1700 nm.

The skilled person will be aware of emitters that absorb and/or emit electromagnetic radiation in the near infra-red II window (NIR-II) and/or in the near infra-red III (NIR-III) window. Exemplary emitters include fluorescent dyes which include organic dyes; quantum dots; and downconversion nanoparticles (DCNPs) for example that emit in the NIR III window.

Accordingly in some embodiments the emitter is:
a) a fluorescent dye;
b) a quantum dot; and/or
c) a downconversion nanoparticle (DCNPs).

The skilled person will be aware of fluorescent dyes that absorb or emit in the NIR-II range. For example, a non-limiting list of fluorescent dyes that are considered to be appropriate are organic dyes including, IR-E1050, and Indocyanine green (ICG). Examples of quantum dots include Inorganic emitters including Ag₂S QDs. In some preferred embodiments the fluorescent dye is selected from, IR-E1050 Indocyanine green (ICG).

The skilled person will appreciate that most fluorescent dyes that are suitable for use with the present invention absorb in the NIR-I region, and emit in the NIR-II region. However, emitters such as downconversion nanoparticles (DCNPs) that can absorb in the NIR-II and emit in the NIR-III are also suitable for use with the present invention and are expected to give deeper penetration through biological tissue due to the reduced effects of biological tissue on both the light that is used to excite the fluorophore, and also the light emitted from the fluorophore.

The ability to conjugate emitters such as fluorescent dyes to nanostructure and nanoparticles such as those of the present invention are known to the skilled person and described in the Examples, for example through the use of a spacer such as thiol-PEG-amine (M_{w}=7500).

The skilled person will appreciate that the nanostructure is designed and tuned so that the strength of the fluorescence emitted from the emitter is enhanced via the phenomenon of metal enhanced fluorescence. It is well known that the distance between the emitter, for example a fluorophore, and the metal nanoparticle dictates whether fluorescence enhancement or quenching will occur. If the spacing between the two materials is too small (less than 5 nm), then the plasmons induced by the irradiation of light are trapped at the metal-emitter interface, resulting in their dissipation as heat and thus quenching of the fluorescence²⁰. If the distance is too large, the intensity of the electromagnetic field felt by the emitter is too weak and little to no enhancement is observed²⁹.

Accordingly, in preferred embodiments, the nanostructure of the invention is such that exposing the nanostructure to light induces the formation of one or more surface plasmons within a portion of the nanostructure, for example wherein the surface plasmon forms on the surface of the metallic material that coats the nanostructure. For example in preferred embodiments the light is selected from the group comprising or consisting: near-infra-red I (NIR) light, NIR-II light, and NIR-III light, or any combination thereof. Commonly used lasers that are suitable for use with the present invention are an 808 nm, 980 nm and 1024 nm laser.

The core of the nanoparticle may take any shape. In producing the nanostructures, the core is generally formed first, with the outer coat of plasmonic metallic material added afterwards. In some embodiments a cross section of the core substantially describes a circle, a square, triangle or a rectangle. In some embodiments a cross section of the core is a circle, a square, a triangle or a rectangle. The core may also have an irregular shape, for example may be a star-shaped structure comprising a plurality of spikes.

In some embodiments, the 3D shape of the core is substantially spherical, substantially hexahedral, substantially cuboid, substantially rectangular cuboid, triangular prism, pyramidal, or substantially cylindrical—for example taking the configuration of a tube or a nanorod. In some embodiments, the 3D shape of the core is spherical, hexahedral, cuboid, rectangular cuboid, triangular prism, pyramidal, or cylindrical or conal or rod shaped.

As mentioned elsewhere herein, a key feature of the nanostructure of the invention is the dielectric core. Some known nanostructures comprise dielectric material elsewhere in the nanostructure, but have a metallic core. Other known nanostructures are made of a solid metallic material. However, the inventors of the present invention have surprisingly found that in fact a dielectric core coated with a metallic plasmonic material has a significantly increased scattering cross-section which contributes to their excitation enhancement, and performed better than solid gold nanostructures. The effect of the dielectric core is considered to outweigh the contribution of the longer and pointier spikes of a solid gold nanostar—see the Examples, where a nanostar comprising a dielectric core but shorter and fatter spikes had improved properties over a solid gold nanostar with longer and pointier spikes, which on the face of it would, prior to the present invention, have been considered to have superior properties.

The dielectric material may be any dielectric material. For example, in some embodiments the dielectric material is a solid, for example in some embodiments the dielectric material is a solid polymer, for example a polymer selected from the group comprising or consisting: polystyrene, silica or any combination thereof. In preferred embodiments the dielectric core is polystyrene.

In some embodiments, the dielectric core is a liquid.

In other embodiments, the dielectric material is a gas, for example is air or dry air. Where the dielectric material is a gas, the dielectric core may be considered to be or termed a hollow core. Air, for example the ambient air present during formation of the nanostructures, is a well-known dielectric material. Other useful dielectric gas materials are considered to be Ammonia, Carbon dioxide, Sulphur hexafluoride (SF6), Carbon Monoxide, Nitrogen, Hydrogen.

To be clear the nanostructure does not comprise a core that is a metal. The core is a dielectric core or a hollow core.

The nanostructure comprises a metallic plasmonic material that coats, or covers, or substantially coats or covers the dielectric core. Suitable metallic plasmonic materials will be known to the skilled person, and includes, for example the group comprising or consisting: a noble metal or a salt thereof; a base metal or a salt thereof; gold or a salt thereof; silver or a salt thereof; copper or a salt thereof; aluminium or a salt thereof; or any combination thereof.

In some preferred embodiments, the metallic plasmonic material is:
a) gold;
b) silver;
c) copper;
d) aluminium; or
e) an alloy of any of gold, silver, copper and/or aluminium.

The spectral properties of the nanostructures of the invention depend largely on the size, shape and material that the nanostructure is made from, and importantly, the aspect ratio of the spikes. Each of these parameters is within the skill of the skilled person to optimise. For example, the optical properties of the nanostructures can be widely tuned, as their LSPR peak position and electric field enhancement depend largely on their size, number of spikes, spike length and aspect ratio. The thickness of the plasmonic metallic material can also be modified to tune the optical properties of the nanostructure.

Accordingly in some embodiments the average diameter (a) of the metallic material-coated nanostructure is:
between 1 nm to 1000 nm, for example between 50 nm to 900 nm; 100 nm to 800 nm; 200 nm to 700 nm; 300 nm to 600 nm, 400 to 500 nm; and/or
at least inm, 50 nm, 100 nm, 200 nm, 300 nm, 400 nm, 500 nm, 600 nm, 700 nm, 800 nm, 900 nm and 1000 nm; and/or
less than 1000 nm, 900 nm, 800 nm, 700 nm, 600 nm, 500 nm, 400 m, 300 nm, 200 nm, 100 nm, 50 nm, 25 nm, or 5 nm, or inm; and/or
between 50 nm to 100 nm.

The diameter (a) is considered to be the longest length of the nanostructure, and where the nanostructure comprises one or more spikes, the diameter (a) is considered to be the longest distance across the nanostructure from spike-end to spike-end.

As described elsewhere herein the nanostructures of the invention have use in in vivo methods, such as live in vivo imaging during surgery, for example to remove a tumour. Preferably, when used in vivo, the nanostructures should not be able to cross the blood brain barrier. Accordingly in some embodiments, for example where the nanostructures are to be used in vivo, the nanostructures should be greater than 5 nm in size, since nanostructures below this size are likely to cross the blood brain barrier.

In certain in vivo applications it Is considered beneficial if the nanostructures are able to “leak” out of the capillaries that surround a tumour. It known that the capillaries around a tumour are particularly “leaky”, so that nanostructures administered into the blood stream pass through the capillaries in the vicinity of the tumour and accumulate. The accumulation of the nanoparticles can be visualised by virtue of the emitter, such as a fluorescent dye, aiding in such things as tumour removal. For the nanostructures to pass through the capillary walls, the size of the nanoparticles should be between 100 nm-150 nm.

Accordingly, in some embodiments the nanostructures of the invention have a diameter of 5 nm or greater, for example of at least 5 nm, 6 nm, 7, 8, 9, 10, 15, 20, 25, 30, 40, 50, 100 nm, 200 nm, 300 nm, 400 nm, 500 nm, 600 nm, 700 nm, 800 nm, 900 nm or at least 1000 nm.

In some embodiments the nanostructures have a diameter of 150 nm or less, for example of less than 145 nm, 140 m, 135, 130, 125, 120, 115, 110, 105, 100, 95, 90 nm or less.

In some embodiments the nanostructures have a diameter of 5 nm or greater and of 150 nm or less i.e. between 5 nm and 150 nm.

In some embodiments the nanostructures have a size or diameter such that crossing of the blood brain barrier is prevented whilst passing through the capillaries around a tumour occurs.

As described elsewhere herein, the size of the core can be varied to tune the optical properties of the nanostructures. In some embodiments the average diameter (b) of the core (for example the spherical core, cuboid core, or rod or cylindrical shaped core as described above) is:
between 1 nm to 1000 nm, for example between 50 nm to 900 nm; 100 nm to 800 nm; 200 nm to 700 nm; 300 nm to 600 nm, 400 to 500 nm; and/or
at least 1 nm, 50 nm, 100 nm, 200 nm, 300 nm, 400 nm, 500 nm, 600 nm, 700 nm, 800 nm, 900 nm and 1000 nm; and/or
less than 1000 nm, 900 nm, 800 nm, 700 nm, 600 nm, 500 nm, 400 m, 300 nm, 200 nm, 100 nm, 50 nm, 25 nm, or 5 nm, or 1 nm.

In some embodiments the longest length of the core (for example where the core is rectangular, or tubular or cylindrical) Is:
between 1 nm to 1000 nm, for example between 50 nm to 900 nm; 100 nm to 800 nm; 200 nm to 700 nm; 300 nm to 600 nm, 400 to 500 nm; and/or
at least 1 nm, 50 nm, 100 nm, 200 nm, 300 nm, 400 nm, 500 nm, 600 nm, 700 nm, 800 nm, 900 nm and 1000 nm; and/or
less than 1000 nm, 900 nm, 800 nm, 700 nm, 600 nm, 500 nm, 400 m, 300 nm, 200 nm, 100 nm, 50 nm, 25 nm, or 5 nm, or 1 nm.

The plasmonic metallic material can have any thickness. For example in some embodiment plasmonic metallic material (c) has a maximum average thickness of:
at least 1 nm, 2, 3, 4, 5, 6, 7, 8, 9, 10, 11, 12, 13, 14, 15, 20, 30, 40, 50, 60, 70, 80, 90, 100, 150, 200, 250, 300, 350, 400, 450, 500, 550, 600, 650, 700, 750, 800, 850, 900, 950 or at least 1000 nm; and/or
less than 1000 nm, 950, 900, 850, 800, 750, 700, 650, 600, 550, 500, 450, 400, 350, 300, 250, 200, 150, 100, 90, 80, 70, 60, 50, 40, 30, 20, 15, 14, 13, 12, 11, 10, 9, 8, 7, 6, 5, 4, 3, 2 or 1 nm; and/or
between 1 nm to 1000 nm, 2 to 950, 3 to 900, 4 to 850, 5 to 800, 6 to 750, 7 to 700, 8 to 650, 9 to 600, 10 to 550, 11 to 500, 12 to 450, 13 to 400, 14 to 350, 15 to 300, 20 to 250, 30 to 200, 40 to 150, 50 to 100, 60 to 90, 70 to 80 nm.

As described elsewhere herein, the dielectric core can take any shape. Similarly, the overall shape of the nanostructure can take any shape. Exemplary advantageous shapes include substantially star-shaped, substantially spherical; substantially hexahedral; substantially cuboid; substantially cylindrical; or any combination thereof; or star-shaped, spherical; hexahedral; cuboid; cylindrical; or any combination thereof. The term “star-shaped” in the context of nanoparticles of the type described herein is a term known in the art.

Since the nanostructures of the invention comprise at least one spike, for example a plurality of spikes along the lines of known nanostars, in some embodiments of the invention it is considered reasonable to term the nanostructures of the invention as:
a) nanostars with a spherical or substantially spherical dielectric core;
b) nanostars with a cuboid or substantially cuboid dielectric core;
c) nanostars with a rectangular cuboid or substantially rectangular cuboid dielectric core;
d) nanostars with a triangular prism or substantially triangular prism dielectric core;
e) nanostars with a pyramidal or substantially pyramidal dielectric core;
f) nanostars with a cylindrical or substantially cylindrical dielectric core;
g) nanostars with a tubular or substantially tubular dielectric core;
h) nanostars with a rod-shaped or substantially rod-shaped dielectric core.

As described elsewhere herein, the dielectric core may be solid, for example a solid dielectric material such as polystyrene; or may be hollow and may be filled with air.

Accordingly the nanostructures of the invention may be considered to be, in some embodiments:
a) nanostars with a hollow spherical or substantially spherical core;
b) nanostars with a hollow cuboid or substantially cuboid core;
c) nanostars with a hollow rectangular cuboid or substantially rectangular cuboid core;
d) nanostars with a hollow triangular prism or substantially triangular prism core;
e) nanostars with a hollow pyramidal or substantially pyramidal core;
f) nanostars with a hollow cylindrical or substantially cylindrical core;
g) nanostars with a hollow tubular or substantially tubular core;
h) nanostars with a hollow rod-shaped or substantially rod-shaped core.

In preferred embodiments the nanostructure of the invention is star-shaped or substantially star-shaped.

In preferred embodiments, the nanostructure comprises a plurality of spikes extending from the surface of the metallic coating.

It is preferred that the spikes are made of the same plasmonic metallic material as that which covers or substantially covers the dielectric core.

In some embodiments the spikes are made of a different plasmonic metallic material as that which covers or substantially covers the dielectric core.

Preferably the surface of the spikes is contiguous with the surface of the metallic coating.

It will be clear that in some embodiments the spikes constitute part of the metallic coating.

The tip diameter of the spikes may be tuned so as to affect the optical properties of the nanostructures. In some embodiments the average diameter (a) of the metallic material-coated nanostructure includes the spikes.

The spikes may be of any length, for example may be of any length:
a) between 20 to 60 nm; for example between 25 nm and 55 nm, 30 nm and 50 nm, 35 nm and 45 nm;
b) of at least 20 nm, for example at least 20, 25, 30, 35, 40, 45, 50, 55, or at least 60 nm; and/or
c) of less than 60 nm, for example less than 60 nm, 55, 50, 45, 40, 35, 30, 25, 20 nm.

The base of the spikes (i.e. the region of the spike that contacts the core) may be of any diameter, for example may be of a diameter of:
a) between 20 to 60 nm, for example between 25 to 55, 30 to 50, 35 to 45 or 40 nm;
b) at least 20 nm, for example at least 25, 30, 35, 40, 45, 50, 55, 60 nm; and/or
c) less than 60 nm, for example less than 55, 50, 45, 40, 35, 30, 25 or less than 20 nm.

The tip of the spike (i.e. the region of the spike distal to the core) can be of any diameter, for example can have a diameter of:
a) between 4 nm to 38 nm, for example between 6 to 36, 8 to 34, 10 to 32, 12 to 30, 14 to 28, 16 to 26, 18 to 24, 20 to 22 nm;
b) at least 4 nm, for example at least, 6, 8, 10, 12, 14, 16, 18, 20, 22, 24, 26, 28, 30, 32, 34, 36, or at least 38 nm;
c) less than 38 nm, for example less than 36, 34, 32, 30, 28, 26, 24, 22, 20, 18, 16, 14, 12, 10, 8, 6, or less than 4 nm.

A spike with a tip with a diameter of 4 nm is considered to be very sharp; a tip with a diameter of 10 nm is considered to have a medium level of sharpness, and a tip with a diameter of 38 nm is considered to have low sharpness.

In preferred embodiments the nanostructure has a plurality of spikes with a tip diameter of less than 10 nm, for example 8, 6, or less than 4 nm so that the spike is considered to be sharp.

In some embodiments the nanostructure has a plurality of spikes which are considered to have different levels of sharpness, for example may comprise spikes with a tip diameter of around 4 nm, for example between 4 nm-9 nm, and may also comprise spikes with a tip diameter of between 10 nm to 30 nm which have a medium sharpness. and may comprise spikes with a tip diameter of between 31 nm and 38 nm.

The aspect ratio is considered to be an important factor In the optical properties of the nanostructure, and is considered to give a measure of the “sharpness” of the spikes and is defined as the length of the spike over the base diameter of the spike.

A spike with an aspect ratio greater than 1.2 is considered to be a sharp spike; a spike with an aspect ratio of around 1 or 1 is consider to be a spike with medium sharpness; a spike with an aspect ratio of less than 1 is considered to be a spike with low sharpness.

In preferred embodiments, the nanostructure of the invention comprises at least one spike, or comprises a plurality of spikes, that have an aspect ratio of greater than 1.2. In some embodiments, the nanostructure of the invention comprises at least one spike, or comprises a plurality of spikes, that have an aspect ratio of 1.

In some embodiments, the nanostructure of the invention comprises at least one spike, or comprises a plurality of spikes, that have an aspect ratio of less than 1.

As described elsewhere herein, the nanostructures have particular utility in the live imaging of internal structures in a subject, for example in a human. For example in some advantageous embodiments the nanostructures are used to aid visualisation of a tumour by a surgeon during tumour removal. There are two main mechanisms by which the nanostructures may accumulate around a tumour so as it enhance visibility.

The first is passive accumulation. This method uses the increased leakiness of capillaries in and around a tumour. Administration of the nanostructures into the blood stream means that the nanostructures leak out of the blood into and around the tumour, in contrast to non-tumorous or non-cancerous tissue.

The second method is an active targeting method. This method uses a targeting moiety, for example an antibody or antigen binding fragment thereof, that is conjugated to the nanostructure, and which has binding specificity for a tumour associated antigen or a tumour neoantigen. This results in specific accumulation of the nanostructures in and around the tumorous tissue.

Nanostructures with target binding agents are also useful for in vitro diagnostics and biosensing methods, as described elsewhere herein.

Accordingly in some embodiments the nanostructures of the invention also comprise a target binding agent capable of binding specifically to a target. In some embodiments the target binding agent is:
a protein, for example a protein selected from the group comprising or consisting: an antibody or antigen binding fragment thereof; or
a nucleic acid, for example an aptamer.

The target may be a target present on a tumour cell or tumour tissue. For example the target may be:
a) a tumour associated antigen (TAA), for example a TAA selected from the group comprising or consisting: alpha-actinin-4 (ACTN4; optionally UniProtKB—043707); AP2S1 (optionally UniProtKB—P53680); BBX (optionally UniProtKB—Q8WY36); BRAF (optionally UniProtKB—P15056); BCR-ABL fusion; CTNNB1 (optionally UniProtKB—P35222); CASP5 (optionally UniProtKB—P51878); CASP8 (optionally UniProtKB—Q14790); CDC27 (optionally UniProtKB—P30260); CDK12 (optionally UniProtKB—Q9NYV4); CDK4 (optionally UniProtKB—P11802); CDKN2A (optionally UniProtKB—P42771); CLPP (optionally UniProtKB—Q16740); UBXN11 (optionally UniProtKB—Q5T124); CSNK1A1 (optionally UniProtKB—P48729); dek-can fusion protein; EFTUD2 (optionally UniProtKB—Q15029); EEF2 (optionally UniProtKB—P13639); ETV6-AML1 fusion protein; FLT3 (optionally UniProtKB—P36888); FN1 (optionally UniProtKB—P02751); FNDC3B (optionally UniProtKB—Q53EP0); GAS7 (optionally UniProtKB—060861); GPNMB (optionally UniProtKB—Q14956); HAUS3 (optionally UniProtKB—Q68CZ6); HLA-A (optionally UniProtKB—P04439); HSDL1 (optionally UniProtKB—Q3SXM5); HSPA2 (optionally UniProtKB—P54652); KRAS (optionally UniProtKB—P01116); KIAAO205; LDLR-fucosyltransferaseAS fusion protein; HHAT (optionally UniProtKB—Q5VTY9); MATN1 (optionally UniProtKB—P21941); ME1 (optionally UniProtKB—P48163); TRAPPC1 (optionally UniProtKB—Q9Y5R8); MUM-3; MYO1B (optionally UniProtKB—043795); NRAS (optionally UniProtKB—P01111); PAPOLG (optionally UniProtKB—Q9BWT3); NFYC (optionally UniProtKB—Q13952); OGT (optionally UniProtKB—015294); OS9 (optionally UniProtKB—Q13438); TP53 (optionally UniProtKB—P04637); pml-RARalpha fusion protein; PPP1R3B (optionally UniProtKB—Q86XI6); PRDX5 (optionally UniProtKB—P30044); PTPRK (optionally UniProtKB—Q15262); UBR4 (optionally UniProtKB—Q5T4S7); SIRT2 (optionally UniProtKB—Q8IXJ6); SNRPD1 (optionally UniProtKB—P62314); SYT-SSX1 or —SSX2 fusion protein; TGFBR2 (optionally UniProtKB—P37173); TPI1 (optionally UniProtKB—P60174); (optionally UniProtKB—).

In some embodiments the target is a tumour neoantigen. The skilled person will recognise tumour neo antigens. Some neoantigens are common to particular types of cancers across different individuals. Some neoantigens are subject specific. The skilled person has the means to identify particular neoantigens, for example by analysing a biopsy of the tumour. Once a neoantigen has been identified, an appropriate target binding agent may be conjugated to a nanostructure of the invention, so that the nanostructure is targeted to sites in the subject that express the neoantigen. See for example Zhang et al Front. Immunol., 16 Apr. 2021| https://doi.org/10.3389/fimmu.2021.672356 and Han et al Front. Cell Dev. Biol., 30 Jul. 2020| https://doi.org/10.3389/fcell.2020.00728

The target is typically associated with a tumour or a cancer. It will be understood that the nanostructures of the present invention have particular utility in the visualisation of solid cancers, rather than blood-borne cancers.

In some embodiments the tumour or cancer is selected from the group comprising or consisting of: triple negative breast cancer, non triple-negative breast cancer, colon cancer, lung cancer, pancreatic cancer, prostate cancer and ovarian cancer. Accordingly, in some embodiments the target is associated with a cell, for example a cancer cell, for example a cancer selected from the group comprising or consisting of: triple negative breast cancer, non triple-negative breast cancer, colon cancer, lung cancer, pancreatic cancer, prostate cancer and ovarian cancer.

For in vitro diagnostic and biosensing purposes, the target may be any target that may be present in a sample obtained from a subject. For example may be an analyte that may be present in a blood sample. For example in some embodiments the target may be target associated with a pathogen, for example a bacterial cell, a virus or a parasite. The skilled person will appreciate that target binding agents, for example antibodies, can be designed so as to have binding specificity to a whole range of different agents and designing such target binding agents is within the skill of the skilled person.

It is also considered that the nanostructures of the present invention may also be used to visualise or detect other growths, such as fibromas and other benign tumours.

In some embodiments, for example in some embodiments where the nanostructure is used to enhance bioimaging during surgery, the target, i.e. the target towards which the target binding agents is directed, is associated with the extracellular matrix or is in the extracellular milieu, for example wherein the extracellular matrix or extracellular matrix is the extracellular matrix or extracellular milieu of a tumour, for example a tumour selected from the group comprising or consisting of: triple negative breast cancer, non triple-negative breast cancer, colon cancer, lung cancer, pancreatic cancer, prostate cancer and ovarian cancer.

As described elsewhere herein, the distance between the emitter such as a fluorophore and the surface of the plasmonic metallic material that coats the dielectric core is considered to be important in determining whether MEF will occur or not. The skilled person is aware of this and is able to select appropriate linker or spacer agents that link the emitter to the nanoparticle surface. For example in some embodiments the linker or spacer is PEG or PSS.

In some embodiments the linker or spacer is such that the emitter, for example a fluorescent dye is positioned at an appropriate distance for the plasmonic metallic material coated surface of the nanostructure. In some embodiments the linker or spacer is chosen so that the emitter for example a fluorescent dye is at least 5 nm from the surface of the nanostructure.

In some embodiments the spacer or linker is selected from the group comprising or consisting of: a carbodimide linker; a polyethylene glycol (PEG) linker or PVP.

The nanostructures of the present invention are considered to be capable of significantly enhancing the fluorescence of an emitter in the NIR-II region. In some embodiments the nanostructures of the present invention are capable of enhancing fluorescence of an emitter, for example a fluorophore when the brightness of said fluorophore in the presence of the nanostructure compared to in the absence of the nanostructure, optionally wherein the increase in intensity is at least a 1%, 2%, 5%, 10%, 15%, 20%, 25%, 30%, 40%, 50%, 60%, 70%, 80%, 90%, 100%, 150%, 200%, 250%, 300%, 350%, 400%, 450%, 500%, 600%, 700%, 800%, 900%, 1000%, 2000%, 3000%, 4000%, 5000%, or 6000% increase in intensity.

In some instances it may be appropriate that the nanostructure of the invention comprises additional emitters, such as additional fluorescent dyes, for example one or more fluorescent dyes in a spectral region other than the NIR-II. Accordingly in some embodiments the invention provides a nanostructure of the invention that comprises at least one additional emitter, for example at least 1, 2, 3, 4, 5 or at least 6 additional emitters, where the at least one additional emitter that absorbs and/or emits electromagnetic radiation in a spectral region other than the near infra-red II window (NIR-II).

It will be appreciated that the nanostructures of the invention can be used in various methods, for example the invention provides a method of:
a) metal enhanced fluorescence;
b) plasmon enhanced fluorescence; and/or
c) enhancing the fluorescence signal emitted by a fluorophore
   wherein the method comprises using the nanostructure according to any of the preceding claims, wherein the nanostructure is capable of enhancing fluorescence of the emitter that absorbs and/or emits electromagnetic radiation in the near infra-red II window (NIR-II).

As described elsewhere herein, the nanostructures may comprise more than one emitter, for example a fluorescent dye that absorbs and/or emits electromagnetic radiation in a spectral region other than the near infra-red II window (NIR-II). Accordingly, in some embodiments the invention provides a method of:
a) metal enhanced fluorescence;
b) plasmon enhanced fluorescence; and/or
c) enhancing the fluorescence signal emitted by a fluorophore
   wherein the method comprises using the nanostructure according to any of the preceding claims, wherein the nanostructure is capable of enhancing fluorescence of at least two different emitters, for example at least three, at least four, at least five, or at least six different emitters (e.g. fluorophores) wherein one emitter absorbs and/or emits electromagnetic radiation in the near infra-red II window (NIR-II) and one emitter absorbs and/or emits in a spectral region other than the near infra-red II window (NIR-II).

Preferences for the level of enhancement of fluorescence are as described elsewhere herein.

For example in some embodiments the invention provides an in vivo method.

As described herein a preferred method for which the nanostructures of the invention may be used is the enhanced resolution in situ fluorescence imaging of biological tissue for preclinical and clinical applications for real-time visualisation, for example of cancerous tumours during surgery to remove all or part of the tumour. Once administered to a subject, or into a sample (e.g. and in vitro organoid) for example into the blood stream, the nanostructures will accumulate around the tumour. Excitation with light of the appropriate wavelength, and detection of the emitted light with an appropriate short-wave infrared (SWIR) cameras capable of receiving the necessary wavelengths, allows real time, accurate visualisation of the tumour. Since the nanostructures of the present invention use emitters that emit in the NIR-II, and which are less susceptible to background fluorescence and absorbance by the tissue, the images that the surgeon sees are much brighter with a greater contrast. This means that tumours can be removed much more successfully with reduced damage to the surrounding tissue and reduced potential for leaving behind some tumorous tissue.

Accordingly in some embodiments the invention provides a method of enhanced resolution in situ fluorescence imaging of biological tissue for preclinical and clinical applications wherein the method comprises administration of nanostructures according to the invention.

In some embodiments the invention provides a method of enhanced resolution in situ fluorescence imaging of biological tissue for preclinical and clinical applications wherein the method comprises visualisation of nanostructures according to the invention that have been previously administered to a subject.

Preferably the imaging is real-time imaging.

Preferences for the cancer or tumour or fibroma are as described elsewhere herein.

In some embodiments the nanostructures of the present invention may be injected straight into a tumour.

The methods of the invention may be performed in vivo, for example during surgery. In some embodiments the methods of the invention are performed in vitro, for example for imaging a tumour model or an organoid.

In some embodiments the methods further comprises:
a) exposing a portion of the subject or sample to light of at least one first wavelength; and
b) detecting the emission of light of at least one second wavelength from the portion of the subject, wherein the light of the second wavelength is in the NIR-I or NIR-II or NIR-III range, preferably in the NIR-II or NIR-III range.

It will be clear to the skilled person that detection of the emission of light of the at least one second wavelength indicates the presence of the nanostructures of the invention; and
the absence of detection of the emission of light of the at least one second wavelength indicates the absence of the nanostructures of the invention.

The presence or absence of the nanostructures of the invention correlates with the presence or absence of a tumour, in the case of for example passive accumulation of nanostructures that do not comprise a target binding domain; or the presence or absence respectively of a target to which a target binding domain present on the nanostructures can bind.

As described above, the nanostructures may not in some embodiments comprise a target binding domain. In some other embodiments the nanostructures do comprise a target binding domain. Preferences for the target binding domain are as described elsewhere here.

As also described above, the size of the nanostructures for in vivo use is important. Too small and they will cross the blood brain barrier; too large and they may not leak out of the capillaries around the tumour. Preferences for the size of the nanostructures in relation to any of the methods described herein are as described elsewhere herein.

Accordingly the method comprises a method of real-time visualisation of a tumour in a subject where the method comprises administering nanostructures of the invention to the subject, for example administered intravenously or directly into the tumour.

In some embodiments, the nanostructures have already been administered to the subject. In this case the method comprises visualisation of the nanostructures once they have already been administered to a subject.

It will be appreciated that the cancer or tumour or target may be present in any subject, for example may be present in a human subject, a dog, a cat, a horse, cattle, other commercially important animals for example. Preferably the subject is a human. In some instances the subject is a subject that has, or is considered to potentially have, cancer. In some instances the subject is a subject that is part of a routine screening programme.

It will be appreciated that any method of using the nanostructures of the invention requires the nanostructures, whether they are surrounding a tumour in vivo, or in an in vitro tube, need to be exposed to light of at least a first wavelength. The light of the first wavelength should correspond to an appropriate excitation wavelength for the emitter, such as a fluorescent dye, for example a fluorescent dye that excites or emits in the NIR-II. The methods will also require that the emission electromagnetic radiation emitted from the emitter (for example a fluorophore) be captured, for example by a NIR-camera that can capture NIR-II wavelengths and portray a visual image, for example to a surgeon.

Where the method is a method for aiding real-time visualisation during surgery, the nanostructures may be administered to a subject ahead of surgery, or during surgery.

The nanostructures of the invention may able be used in vitro, for example in a method of in vitro biosensing, for example in a sample that is, or that comprises, blood; or a s liquid biopsy.

When the nanostructures are used in vitro, the size of the nanostructures is considered to be less critical, and the nanostructures may be of any appropriate size. For example, in some embodiments the method comprises the in vitro detection of a target. A target may be any target, for example any target present on a cell, for example on a cancer cell. Preferences for the nature of the target, for example a tumour associated antigen or a tumour neo antigen are as described elsewhere herein.

Such in vitro methods may be used in diagnostics, for example in some embodiments the method may be a method of diagnosing cancer, wherein the nanostructures of the invention comprise a target binding agent as described elsewhere herein capable of binging to a tumour associated antigen or neo antigen. In some embodiments the target binding agent may able to bind to circulating tumour DNA.

In some embodiments the invention provides an in vitro method for determining the presence or level of a component in a sample taken from a subject. The sample may be any sample, for example may be selected from the group comprising or consisting of: blood, plasma, mucus, transudate, urine, milk, phlegm, saliva, bile, semen, tears, pus, sebum, intracellular fluid, interstitial fluid, cerebrospinal fluid, lymphatic fluid, tissue sample, bone sample, bone marrow sample, breast sample, gastrointestinal sample, lung sample, liver sample, pancreatic sample, prostate sample, brain sample, nerve sample, meningeal sample, renal sample, endometrial sample, cervical sample, lymph sample, muscle sample, skin sample, or any combination thereof. In some preferred embodiments the sample is a blood sample or a liquid biopsy.

Preferences for the subject are as described elsewhere herein.

In some embodiments, the in vitro methods described herein comprise:
a) contacting a plurality of the nanostructures of the invention that comprise a target binding domain with a sample so as to allow binding of the target binding domain to the target, if present in the sample;
b) removal of nanostructures that are not bound to the target;
c) exposing the nanostructure to light of at least one first wavelength; and
d) detecting the emission of light of at least one second wavelength from the nanostructure, wherein the second wavelength is in the NIR-I or NIR-II range;
wherein the detection of the emission of light of the at least one second wavelength indicates the presence of the target; and
the absence of detection of the emission of light of the at least one second wavelength indicates the absence of the target.

Preferred in vitro methods are a method for point of care biosensing in NIR II and NIR III windows, involving the use of the nanostructures of the invention.

The invention also provides the nanostructure according to the invention for use in medicine.

The invention also provides a composition comprising the nanostructure (or plurality of nanostructures) according to the invention.

It will be appreciated that in some instances it may be preferable to use a combination of different nanostructures of the invention, perhaps with different fluorophores or different optical properties. Accordingly the invention also provides a composition comprising at least a first plurality of nanostructures of the invention and at least a second plurality of nanostructures, wherein the first and second plurality of nanostructures are different. In some embodiments the composition comprises third, fourth, fifth, sixth, seventh, eighth, ninth, and/or tenth plurality of nanostructures according to the invention.

It will be clear that reference to a particular plurality of nanostructures implies that each nanostructure in the plurality is the same, or at least as similar as can reasonably be possible—for example the nanostructures will have been made by the same method. For example, the core of each nanostructure of the first plurality of nanostructures is composed of the same material as the core of every other nanostructure of the first plurality of nanostructures; and the coating of each nanostructure of the first plurality of nanostructures is composed of the same material as the coating of every other nanostructure of the first plurality of nanostructures.

This is also the case for the other plurality of nanostructures, i.e. each of the nanostructures of the:
second plurality of nanostructures are the same;
third plurality of nanostructures are the same;
fourth plurality of nanostructures are the same;
fifth plurality of nanostructures are the same;
sixth plurality of nanostructures are the same;
seventh plurality of nanostructures are the same;
eighth plurality of nanostructures are the same;
ninth plurality of nanostructures are the same;
tenth plurality of nanostructures are the same;

i.e. the core of each nanostructure of the second, third, fourth, fifth, sixth, seventh, eighth, ninth, and/or tenth plurality of nanostructures is composed of the same material as the core of every other nanostructure of the second optionally third, fourth, fifth, sixth, seventh, eighth, ninth, and/or tenth plurality of nanostructures; and the coating of each nanostructure of the second, optionally third, fourth, fifth, sixth, seventh, eighth, ninth, and/or tenth plurality of nanostructures is composed of the same material as the coating of every other nanostructure of the second, optionally third, fourth, fifth, sixth, seventh, eighth, ninth, and/or tenth plurality of nanostructures.

The same concept holds true for other parameters, for example size of core, size of spikes in the context of nanostars.

The composition of the invention may also comprise a pharmaceutically acceptable buffer.

The invention also provides a pharmaceutical composition comprising the nanostructure of the invention.

The invention also provides the composition of pharmaceutical composition for use in medicine.

The invention also provides various methods for producing the nanostructures of the invention.

In one embodiment the method comprises:
a) providing at least one dielectric core;
b) decorating the at least one dielectric core with a plurality of seeds; and
c) Incubating the decorated dielectric core in a growth solution.

In some embodiments, the growth solution comprises an anionic surfactant, for example CTAC. The skilled person will appreciate that the anionic surfactant is toxic and must be washed away prior to use. The sharpness (aspect ratio) of the spikes are controlled by choices of surfactant. For example, the production of spikes with a low sharpness (i.e. a low aspect ratio) on the nanostructures of the invention that comprises one or a plurality of spikes, CTAC surfactant may be used. For the production of medium-sharpness spikes (i.e. medium aspect ratio) on the nanostructures of the invention that comprises one or a plurality of spikes CTAB surfactant may be used. For the production of spikes with a high level of sharpness (i.e. a high aspect ratio) on the nanostructures of the invention that comprises one or a plurality of spikes CTAC and NaBr mixed solution may be used. The Examples describe methods of producing the nanostructures of the invention, but in brief, a seeded dielectric core is added to an exemplary growth solution that contains the above mentioned surfactant (depending on the sharpness required), and Au precursors (HAuCl₄), AgNO3, and ascorbic acid. After ˜ two hours reaction, a faint blue appeared. The synthesised core-shell Au nanostars can be purified by centrifuge and dispersed into water.

In some embodiments the seeds are not of the same metallic material as the plasmonic metallic material that coats or substantially coats the dielectric core. In some embodiments the seeds are of silver, and the plasmonic metallic material is gold.

In some preferred embodiments the dielectric core is polystyrene, for example a polystyrene sphere of 60 nm or 80 nm, the seeds are silver and the plasmonic metallic material is gold, and the nanostructure takes the form of a nanostar.

Incubation of the decorated dielectric core in the growth solution coats the decorated dielectric core in a plasmonic metallic coating.

Preferences for the dielectric core, for example size and material are as described elsewhere herein, as are preferences for the plasmonic metallic material.

In some embodiments the method further comprises the step of (d) functionalising the structures, for example with PEG.

In some embodiments the method further comprises the step of (e) conjugation of an emitter that emits in the NIR-II, for example IR-E1050-COOH and ICG.

The invention also provides a method of tuning the nanostructure of the invention to have particular optical properties, wherein the method comprises modifying at least one property of the nanostructure to enhance the fluorescence intensity of light emitted by at least one emitter bound to the nanostructure, optionally wherein the method comprises modifying at least one property of the nanostructure to enhance the fluorescence intensity of light emitted by a plurality of emitters bound to the nanostructure, optionally wherein each emitter of the plurality of emitters has a peak excitation wavelength (λ_{Ex}) that is different to the peak excitation wavelength (λ_{Ex}) of every other fluorophore of the plurality of emitters.

In some embodiments the at least one property is selected from the group comprising or consisting of:
(i) the size of the nanostructures;
(ii) the shape of the nanostructures;
(iii) the thickness of the coating of the metallic plasmonic material;
(iv) the length of or aspect ratio of the spikes extending from the surface of the coating;
(v) the type of metallic plasmonic material; or
(vi) the dielectric material;
or any combination thereof.

The invention also provides a nanostructure, method, composition, or kit substantially as described herein.

The listing or discussion of an apparently prior-published document in this specification should not necessarily be taken as an acknowledgement that the document is part of the state of the art or is common general knowledge.

Preferences and options for a given aspect, feature or parameter of the invention should, unless the context indicates otherwise, be regarded as having been disclosed in combination with any and all preferences and options for all other aspects, features and parameters of the invention. For example:

The invention provides a nanostructure that comprises a spherical dielectric core of polystyrene with a diameter of 60 nm, seeds of silver and a gold coating that coats the dielectric core and silver seeds, and which also comprises gold spikes, giving the form of a core-shell nanostar and which also comprises the fluorescent dye IR-E1050-COOH conjugated to the nanostructure with PEG;

The invention also provides a nanostructure that comprises a spherical dielectric core of polystyrene with a diameter of 80 nm, seeds of silver and a gold coating that coats the dielectric core and silver seeds, and which also comprises gold spikes, giving the form of a core-shell nanostar, and which also comprises the fluorescent dye ICG conjugated to the nanostructure with PEG.

The Invention also provides a nanostructure of the invention comprising a cuboid dielectric core coated in gold and comprising a plurality of gold spikes;

The invention also provides a hollow nano-tube coated in gold and comprising a plurality of gold spikes.

The invention also provides a method for real-time enhanced visualisation of a tumour during surgery wherein the subject has been administered a plurality of nanostructures that:
a) comprise a spherical dielectric core of polystyrene with a diameter of 60 nm, seeds of silver and a gold coating that coats the dielectric core and silver seeds, and which also comprises gold spikes, giving the form of a core-shell nanostar and which also comprises the fluorescent dye IR-E1050-COOH conjugated to the nanostructure with PEG;
b) comprises a spherical dielectric core of polystyrene with a diameter of 80 nm, seeds of silver and a gold coating that coats the dielectric core and silver seeds, and which also comprises gold spikes, giving the form of a core-shell nanostar, and which also comprises the fluorescent dye ICG conjugated to the nanostructure with PEG.

### FIGURE LEGENDS

FIG. 1: Schematic workflow of MEF probe synthesis whereby core seeds of either gold or silver-decorated polystyrene were placed in a gold growth solution to form the highly branched structures of solid and core-shell nanostars respectively. They were then coated in a layer of thiol/amine functionalised PEG to allow for subsequent binding of the fluorophore and measurement of fluorescence enhancement.

FIG. 2: (a) Normalised extinction spectra of SNS-150 and CSNS-80 overlaid against the normalised emission spectrum of IR-E1050-COOH, (b-e) Transmission electron microscopy (TEM) Images of the (b) 50 nm citrate-stabilised Au seeds, (c) Ag-seeded 80 nm polystyrene (PS) spheres, (d) core-shell nanostars made from 80 nm PS (CSNS-80) and (e) solid nanostars made from 50 nm Au seeds (SNS-150). Scale bars for the micrographs are: (b) 0.2 μm, (c) 100 nm and (d, e) 0.5 μm.

FIG. 3: Photoluminescence (PL) spectra of equal concentrations of IR-E1050-COOH in water (black), conjugated to solid nanostars in solution (blue) and conjugated to core-shell nanostars in solution (red).

FIG. 4: (a, b, d, e) TEM images of the (b) 15 nm citrate-stabilised Au seeds, (b) Ag-seeded 60 nm polystyrene (PS) spheres, (c) normalised extinction spectra of SNS-150 and CSNS-80 overlaid against the normalised emission spectrum of IR-E1050-COOH, (d) solid nanostars made from 50 nm Au seeds (SNS-150) and (e) core-shell nanostars made from 60 nm PS (CSNS-60). Scale bars for the micrographs are: (a) 50 nm, (b, d) 100 nm and (e) 0.5 μm.

FIG. 5: PL spectra of equal concentrations of ICG in water (black), bound to solid nanostars in solution (blue) and core-shell nanostars (red). The inset shows the PL emission of free ICG, zoomed in to show the shoulder at 1000 nm. PL spectra were acquired using an excitation wavelength of 782 nm.

FIG. 6: need to label graphs a) PL spectra of free and enhanced ICG compared to free and enhanced IR-E1050-COOH using S—NS, b) PL spectra of free and enhanced ICG compared to free and enhanced IR-E1050-COOH using CS—NS. Both insets show the zoomed in spectra of IR-E1050-COOH

FIG. 7: Other exemplary nanostructures of the invention. Example silica tube (top) and silica cube as cores for core-shell Au nanostars growth.

FIG. 8: CTAB-free Au Nanotube via silica-templated method

FIG. 9: Other suitable Plasmonic Nanostructure candidates: A) SiO₂@Cu₂O cube; B) Hollow Au cubes.

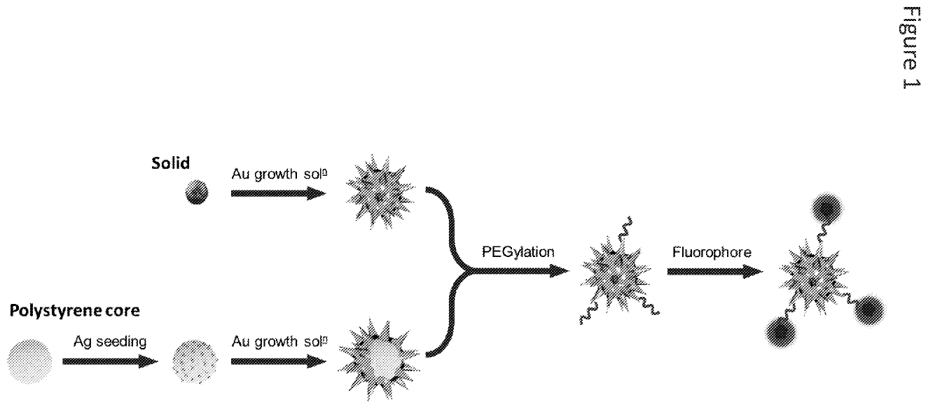

### EXAMPLES

### Example 1

### Synthesis and Characterisation of NIR-II AuNS

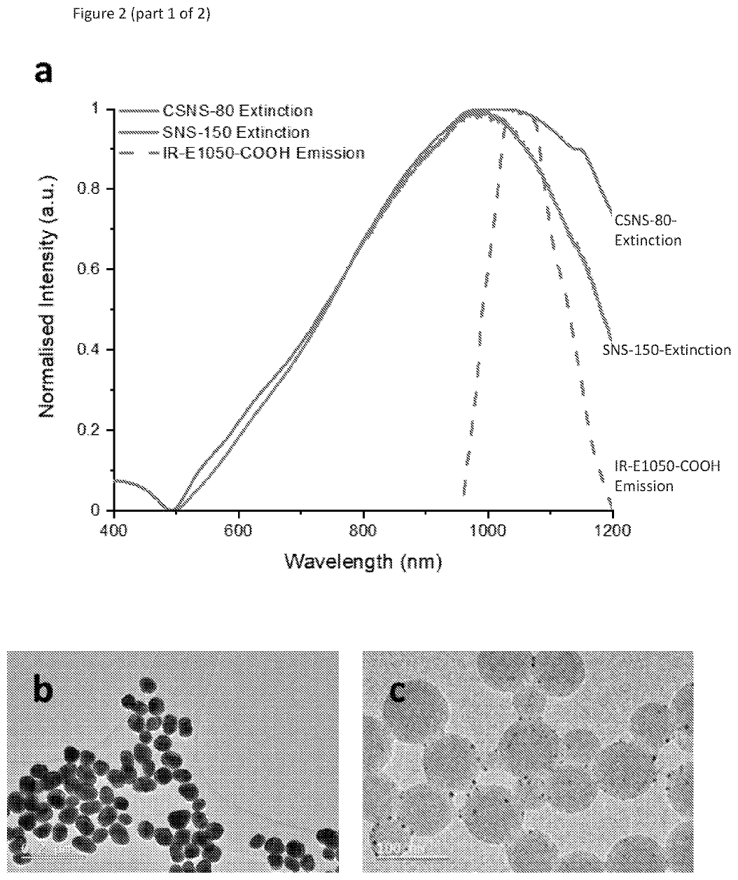

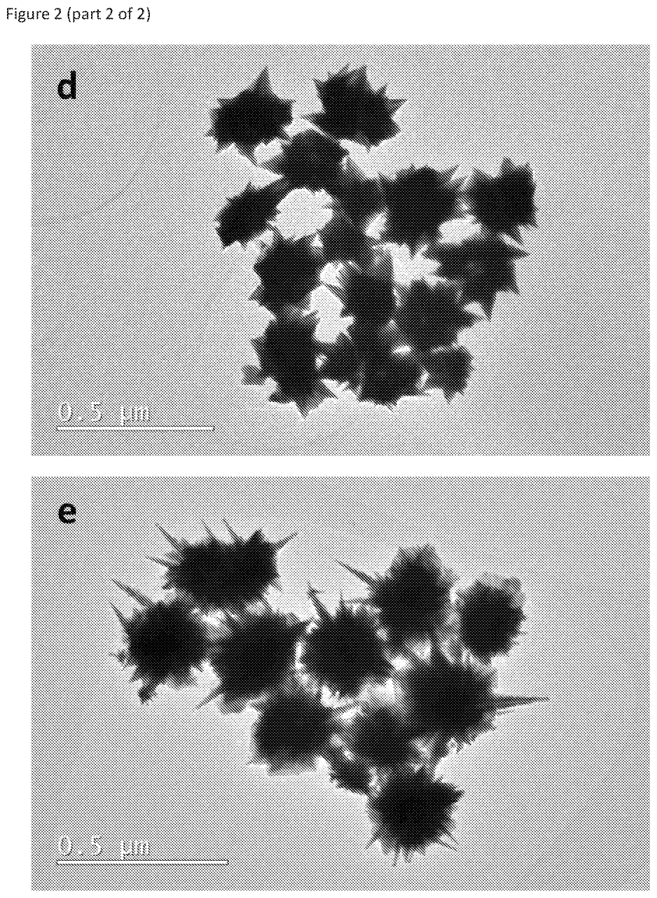

Solid and core-shell Au nanostars were synthesised as per the methods described. For the SNS synthesis, a seed-mediated, surfactant-free method using 50 nm Au seeds (FIG. 2b) was used¹³. This lack of surfactant on the surface of the AuNS simplified further functionalisation with the fluorophore. In the case of the seed-mediated CS-NSs synthesis, however, the anionic surfactant, CTAC, was vital in directing the growth of the branches on the surface of the Ag-decorated polystyrene spheres (FIG. 2c)^{15,26}. This surfactant, while necessary for the nanostar synthesis, is both toxic and not suitable as a linker molecule to conjugate the nanostar to the fluorophore. Therefore, it was necessary to wash the CS-NSs multiple times in order to remove as much of the surfactant as possible before functionalisation could occur.

TEM micrographs of both nanostar types (FIG. 2d, e) show the anisotropic morphology of a spherical core with numerous protruding branches that is typical of AuNSs. Although both S—NS and CS—NS were of a large size (260 nm and 190 nm respectively) and displayed sharp tips at the ends of their branches, the spikes of the S—NS were longer and sharper, while the CS—NS spikes were shorter and fatter. It is at the tips of these branches where, upon illumination with an appropriate wavelength light, electric field enhancements are at their greatest (several orders of magnitude)²⁷. Due to the large degree of branch shape variation, the nanostars display multiple plasmon resonances, leading to the characteristic broadness of their LSPR (FIG. 2a) across the NIR-I and NIR-II.

By adjusting reaction parameters such as the concentrations of Au³⁺, Ag⁺ and AA during the nanostar synthesis, the shape, size and number of spikes present on the particles was altered and thus, so was their LSPR. This ease of tunability enabled the synthesis of S—NS and CS—NS with LSPRs showing intense absorption and scattering between 800 and 1200 nm. These LSPRs overlapped well with the optical properties of the NIR-II fluorophore, IR-E1050-COOH, which was known to have excitation and emission maxima at 800 and 982 nm respectively (FIG. 2a). This spectral overlap was vital in order for MEF to occur.

Functionalisation with PEG

Prior to functionalising the nanostars with the NIR-II fluorophore, a spacer molecule had to first be applied. A thiol-PEG-amine (M_{w}=7500) was used as the spacer between the AuNS and dye for a number of reasons.

Firstly, the thiol (—SH) functionalisation present on one end of the PEG chain enabled attachment of the polymer to the surface of the AuNS, due to the strong binding energy of the gold-thiolate bond. The other end of the chain was functionalised with an amine (—NH₂) group, which provided covalent coupling points to which the carboxylate (—COOH) group of the dye could readily attach to via carbodiimide cross linker chemistry.

Additionally, the overall length of the polymer chain in solution is known to be between 8 and 13 nm²S. It is well known that the distance between the fluorophore and the metal nanoparticle dictates whether fluorescence enhancement or quenching will occur. If the spacing between the two materials is too small, then the plasmons induced by the irradiation of light are trapped at the metal-fluorophore interface, resulting in their dissipation as heat and thus quenching of the fluorophore²⁰. If the distance is too large, the intensity of the electromagnetic field felt by the fluorophore is too weak and little to no enhancement is observed²⁹. Though experimentation, it has been found that for gold nanoparticles, depending on their shape, a distance of 5-20 nm between the particle and dye gives maximum fluorescence enhancement²⁹⁻³¹. Therefore, for this investigation, using the thiol-PEG-amine as a spacer placed the dye at an optimum distance from the AuNS surface, giving rise to enhanced fluorescence.

### Fluorophore Conjugation and Fluorescence Enhancement

Fluorescence enhancement in the NIR-II was investigated using the commercially available dye IR-E1050-COOH. By quantifying the amount of dye attached to the nanostars and preparing suitable controls containing equal concentrations of free dye, the amount of fluorescence enhancement was measured.

IR-E1050-COOH was selected as the fluorophore for this investigation as it exhibits a large Stoke's shift (i.e. It can be excited by NIR-I light and emit in the NIR-II range), is biocompatible and is easily functionalised due to its terminal carboxylate group. Moreover, it has a low quantum yield of 1.9%, meaning that, in order for It to be applied to in vivo imaging, its efficiency would need to be greatly improved, which makes it an ideal candidate to which MEF can be applied.

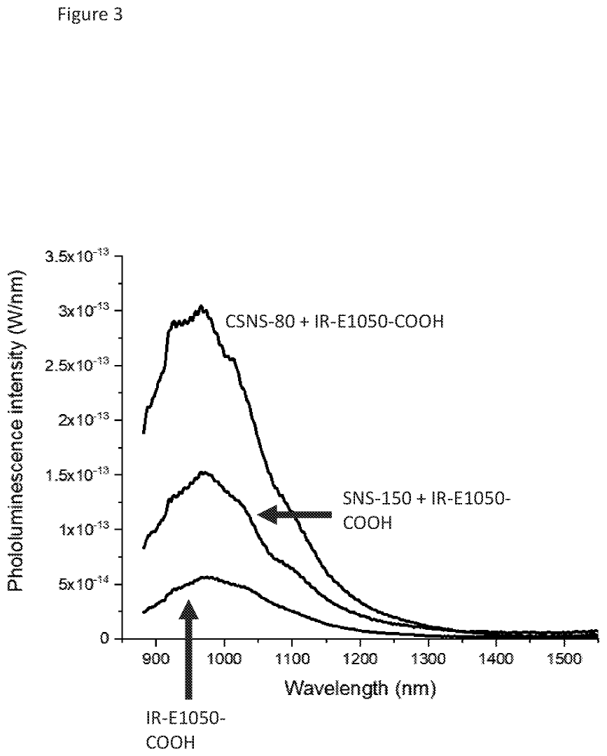

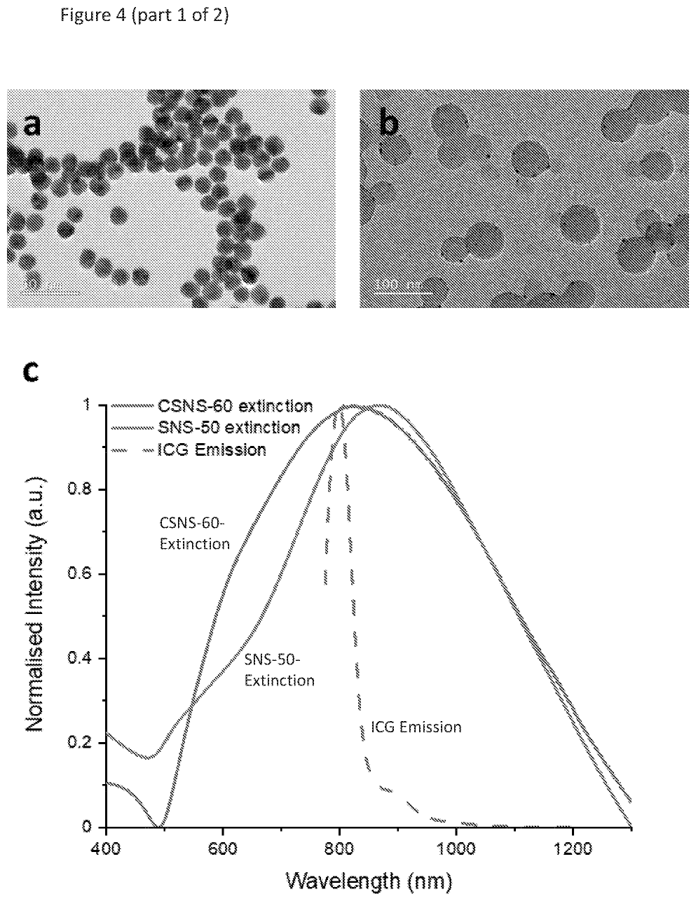

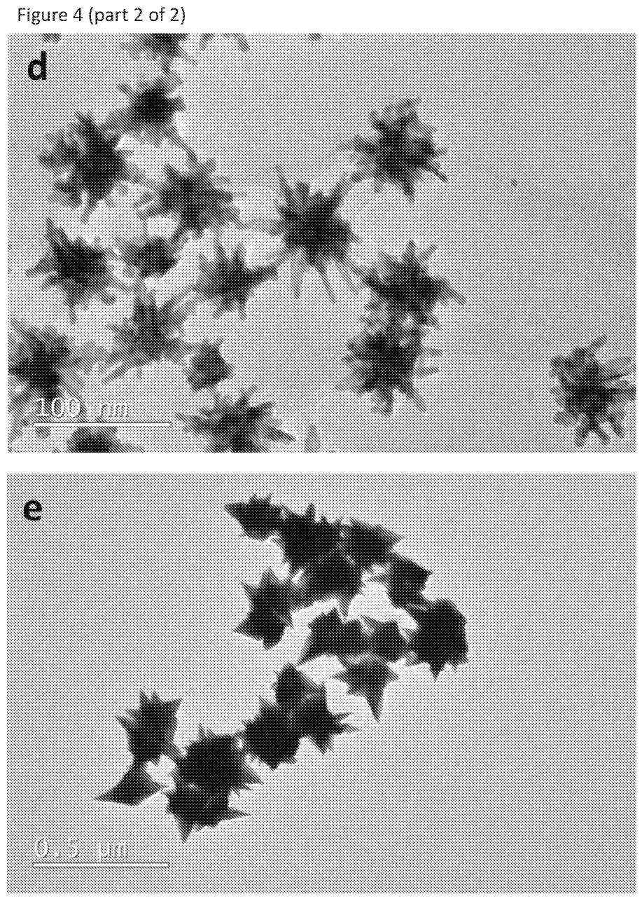

The averaged emission spectra of IR-E1050-COOH bound to S—NS, CS—NS, and free in water are displayed in FIG. 3. The fluorescence enhancement factors (Er) achieved were calculated using the following formula:

$${E}_{f} = \frac{{E}_{dye + AuNs} - {E}_{water}}{{E}_{dye} - {E}_{water}} \qquad ( 1 )$$

where E_{dye+AuNS} and E_{dye} are the fluorescence intensities of the fluorophore on the AuNS and in water, respectively, and E_{water} is the background fluorescence of the water.

TABLE 1
Average fluorescence enhancement factors E_{f} of emission
at 1000 nm measured in solution for IR-E1050-COOH
conjugated to solid and core-shell nanostars.

| Fluorophore | Nanostar | E_{f} at 982 nm |
|---|---|---|
| IR-E1050-COOH | SNS-150 | 3.27 |
| | CSNS-80 | 5.84 |

Table 1 contains a summary of these enhancement factors and show that both AuNS structures display MEF with the dye, with S—NS enhancing 3.27 times and CS—NS enhancing 5.84 times. As seen in FIG. 2, both nanostar morphologies display similar sizes and spectral overlap with IR-E1050-COOH. Although the S—NS possesses sharper tips, enabling higher electric field enhancements than the CS—NS, the CS—NS have a dielectric core, which significantly increases their scattering cross-section and contributes to their excitation enhancement.

### Example 2—Enhancing the NIR-II Tall of a NIR-I Dye

### Nanostar Synthesis and Functionalisation

While the work detailed above shows the great promise of enhancing fluorescence in the NIR-II, it is difficult to progress these fluorescent probes to clinical research, because the dye IR-E1050-COOH, as with all currently available NIR-II dyes, is not FDA approved. The only current FDA-approved NIR dye is ICG, a NIR-I fluorophore with a peak emission at 800 nm. It has been used in numerous MEF studies and has shown significant fluorescence enhancement in the NIR-I^{32,33}. Looking closely at the emission spectrum of ICG, particularly the tall-end emission, a small shoulder at 900 nm and another at 1000 nm can be seen³⁴. Recently, Investigators have started looking at this tail-end emission for NIR-II imaging^{35,36}, as it has been shown to be brighter than equal concentrations of commercially available NIR-II dyes⁸. To our knowledge, although studies of enhancing the fluorescence of ICG in the NIR-I are numerous^{33,37}, there have been no investigations of fluorescence enhancement of ICG in the NIR-II.

To this end, the S—NS and CS—NS previously used to enhance IR-E1050-COOH were redesigned to have their spectral properties overlap with those of ICG, by varying their seed size and concentrations of reagents in their growth solutions. To make the new S—NS, Au seeds (15 nm) were used, while 60 nm PS cores were used to make the new CS—NS. The TEM micrographs show a more marked difference in the morphologies of the S—NS and CS—NS, with the CS—NS having a similar shape to the previous CS—NS, while the S—NS are smaller and have blunter tips than the previous S—NS.

The LSPRs of both new morphologies showed an intense, broad peak between 700 and 1000 nm, overlapping with the excitation (790 nm) and emission (800 nm) peaks of ICG, with the CS—NS having slightly better spectral overlap. Although thiol-PEG-amine was used to coat the nanostars, no crosslinker chemistry was used to conjugate the dye. Instead, electrostatic coupling between the nanostars and ICG was exploited. In aqueous solution at pH 7.4, the exposed NH₂ groups on the PEG are protonated to NH₃⁺, giving the nanostars a positive surface charge, while ICG has a negative charge. Multiple rounds of centrifugation of the nanostar-ICG complexes showed that there was no loss of coupled ICG from the surface of the nanostars.

### Fluorescence Measurements

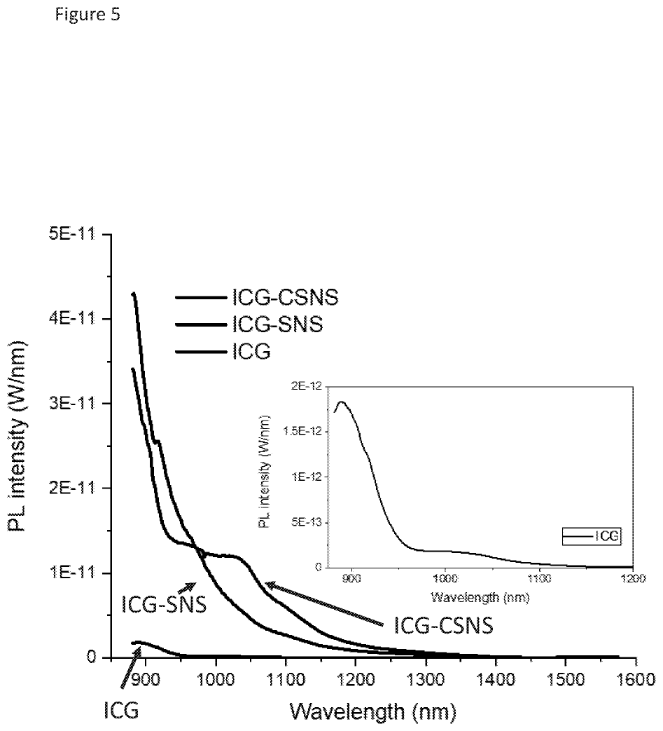

The fluorescence spectra of ICG bound to S—NS and CS—NS are shown in FIG. 5. The fluorescence spectra were collected under identical excitation and detection conditions and their E_{f} (Table 2) were calculated at different wavelengths using Equation 1. The fluorescence intensities at 900 nm and 1000 nm were chosen as these wavelengths are easily detectable by short-wave infrared (SWIR) cameras and can be selectively detected using specialised filters, making them ideal wavelengths for imaging. As shown in Table 2, ICG displayed significantly enhanced fluorescence on both nanostar morphologies. Remarkably, fluorescence enhancement of up to 66 times was achieved in the NIR-II.

Since the magnitude of MEF largely depends on the spectral overlap between the optical properties of the fluorophore and the LSPR of the plasmonic nanostructure-, we attribute the more pronounced shoulder at 1000 nm for CS—NS conjugated ICG to the superior spectral overlap between the nanostars and the ICG, compared to that of the S—NS.

TABLE 2
Average fluorescence enhancement factors E_{f} of
emission at 900 and 1000 nm measured in solution for
ICG conjugated to solid and core-shell nanostars.

| Fluorophore | Nanostar | E_{f} at 900 nm | E_{f} at 1000 nm |
|---|---|---|---|
| ICG | SNS-50 | 18.39 | 55.37 |
| | CSNS-60 | 15.69 | 66.09 |

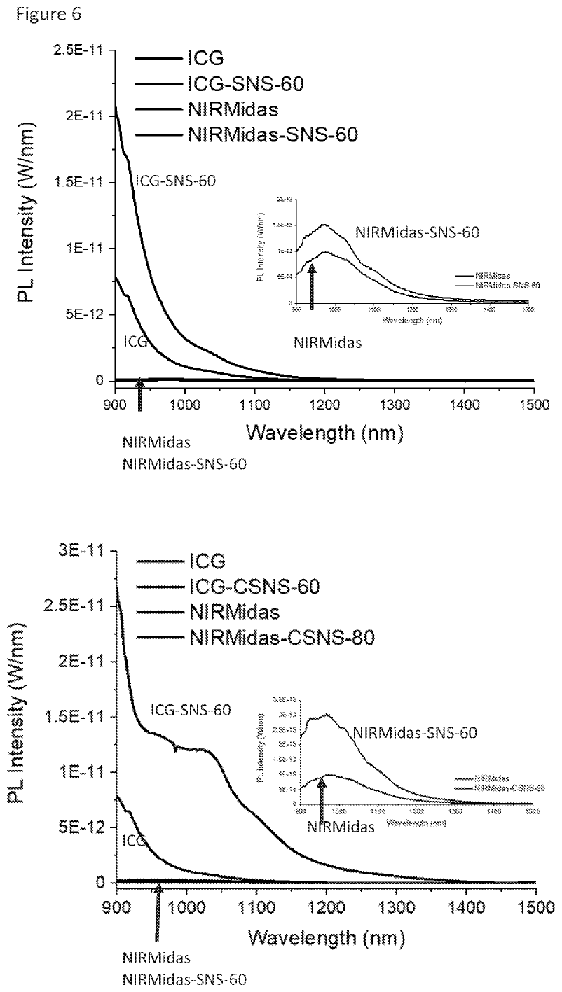

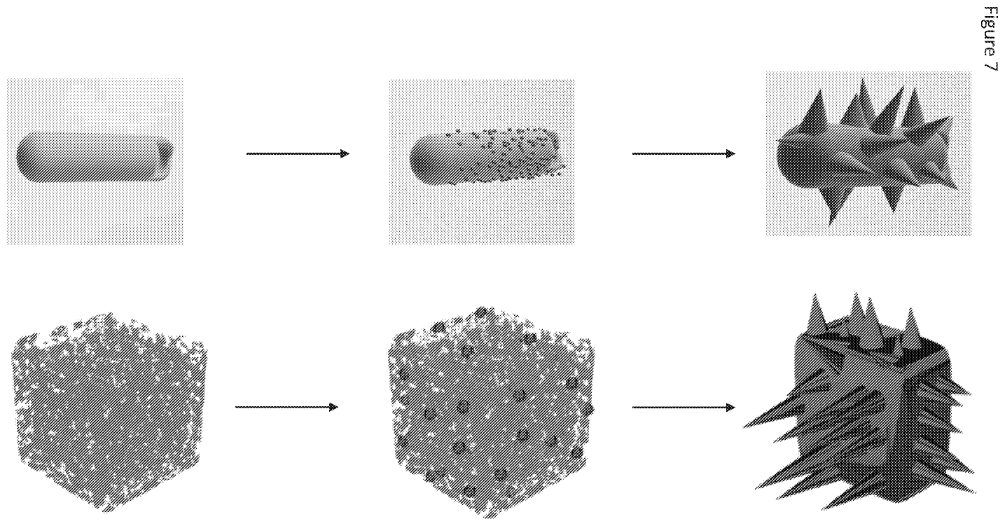

In our results, the highest fluorescence enhancement was observed from ICG-CSNS at 1000 nm, giving 66 times higher fluorescence than that of free ICG in solution and an order of magnitude higher fluorescence than that achieved for an equivalent concentration of IR-E1050-COOH. In fact, we have shown that even the tail-end emission of ICG is still brighter than the enhanced emission of a NIR-II dye (FIG. 6). Adding this to the fact that ICG is already an FDA-approved fluorophore, these nanostar-dye designs hold great promise for translation into clinical use.

### Conclusions

To summarise, we have shown that the NIR-II fluorescence of the clinically approved NIR-I dye ICG can be enhanced using MEF such that it significantly outperforms all known NIR-II fluorophores and may progress faster to the clinic. Four different MEF probes were synthesised and their single-particle fluorescence enhancements were compared. Each probe consisted of NIR fluorescent dyes conjugated to AuNS of different structures and their NIR-II emission was analysed. For the NIR-II dye, fluorescence enhancement of up to 5 times is shown using a core-shell morphology, while a solid AuNS structure yielded enhancement of 3. For the tall emission of the NIR-I dye, enhancement of up to 55 times and 66 times are shown for solid and core-shell morphologies respectively. This work supports the potential of AuNS as future NIR-MEF platforms for developing probes for non-invasive bioimaging applications, where molecular targeting can be used to clearly define tissue boundaries and thus provide surgeons with real-time imaging in the biologically transparent NIR-II window.

### Example 3—Methods

### Solid Nanostar Synthesis

Solid nanostars (S—NS) were synthesised via a well-known seed-mediated method²¹⁻²³.

To make the initial seeds from which the nanostars were grown, 100 mL of a 0.25 mM aqueous HAuCl₄·3H₂O solution was heated to boiling in a 250 mL Erlenmeyer flask under magnetic stirring. Then, to make Au seeds of 15 nm and 50 nm respectively, 1 mL or 0.25 mL of a 3.3% (w/v) aqueous sodium citrate solution was added to the flask under vigorous stirring. After a couple of minutes, the solution appeared a blue-purple colour, which, after approximately 10 minutes, became a stable, bright red colour. The seed solution was then cooled in an ice bath and its volume was made back up to 100 mL with Milli-Q water. The seeds were stable and could be stored long-term at 4° C.

To grow 50 nm solid nanostars (SNS-50), firstly, 200 μL of 15 nm seeds were added to 10 mL of a rapidly stirring 0.1 mM HAuCl₄·3H₂O solution containing 10 μL of 1 M HCl. For the 150 solid nanostars (SNS-150) 300 μL of 50 nm seeds were added to 10 mL of a 0.3 mM HAuCl₄·3H₂O solution with 10 μL of 1 M HCl. Then, 150 μL of 2 M AgNO₃, followed quickly by 50 μL of AA, was added into the stirring solutions. An appearance of a faint blue colour to the solution after 30 seconds indicated the completion of the reaction.

### Core-Shell Nanostar Synthesis

The core-shell nanoparticles were synthesised according to a method previously described for polystyrene(PS) cores^{15,19,24}. Two sizes of PS (60 and 80 nm) were used to make two differently sized core-shell nanostars.

Briefly, the cores were initially decorated with silver seeds, and then placed in a gold growth solution containing surfactant to form the star-shaped shell.

To first coat the PS core particles with silver seeds, 5 mL of a 0.04% solid PS solution (60 or 80 nm) was stirred in a round bottomed flask. Then, 100 μL of an 80 mM [Ag(NH₃)₂]⁺ solution—prepared by the mixing of AgNO₃ and NH₄OH—was slowly added to the gently stirring PS solution and allowed to react for one hour in the dark. To filter out any unreacted silver from the solution, it was centrifuged and washed with water.

The purified PSAu particles were then transferred to a round bottomed flask and the solution was made up to 5 mL with cold water. 50 μL of ice-cold 130 mM NaBH₄ was rapidly added to the vigorously stirring solution and the reaction mixture was left in the fridge overnight to allow decomposition of any unreacted NaBH₄. The seeded PS spheres were then centrifuged, washed with water and redispersed in 5 mL of water.

To grow the Au nanostar shell onto the PS spheres, 10 μL of the seeded spheres were added to a gently stirring growth solution containing 10 mL of 100 mM CTAC solution, 790 μL of 1 M NaBr, 421 μL of 10 mM HAuCl₄·3H₂O, 64 μL of 10 mM AgNO₃ and 67 μL of 100 mM AA. A faint blue colour appeared after 2 hours and the core-shell nanostars were centrifuged and washed before being redispersed in 10 mL of water.

### Nanostar Characterisation

The as-synthesised solid and core-shell nanostars were characterised by optical absorption spectroscopy using an Agilent Cary 5000 UV/Vis-NIR spectrophotometer. Samples were analysed in glass cuvettes from VWR International and their extinction spectra were collected. A baseline correction using Milli-Q water was applied to account for water/cuvette absorption. Transmission electron microscopy was performed using a JEOL JEM-2100Plus with an accelerating voltage of 200 kV. Particle sizes and morphologies were analysed using TEM micrographs and the image processing software ImageJ.

### Nanostar Functionalisation

In order to conjugate the nanostars to the fluorescent dyes, the particles were first functionalised with carboxylate groups. Two separate solutions of 10 mM thiol-PEG-amine and 10 mM methyl-PEG-thiol were prepared and mixed in a 9:1 ratio. 100 μL of this PEG mixture was added to each 10 mL nanostar solution and after 2 hours of gentle shaking, the unattached PEG was centrifuged off and the nanostars were redispersed in 10 mL of water.

### Fluorophore Conjugation

The fluorophores investigated in this study were the commercially available IR-E1050-COOH and ICG.

In the case of IR-E1050-COOH, 5.5 μL of a 1 mg/mL solution of dye was mixed with 8 μL of 10 mM EDC and 8 μL of 25 mM NHS for 15 minutes. Then, 110 μL of PBS was added to the now-activated dye, and the entire mixture was added to 10 mL of nanostar solution. This was gently shaken in the dark overnight, centrifuged and made up to 10 mL, and the supernatant was collected for measurement. ICG was conjugated to the nanostars through electrostatic coupling²⁵. 25 μL of a 0.1 mg/mL dye solution was added to 10 mL of nanostar solution and was gently shaken overnight in the dark, followed by centrifugation and collection of the supernatant for measurement.

### Spectroscopy Measurements

Fluorescence emission spectra of IR-E1050-COOH and ICG were collected in the range of 880-1570 nm using an NS 1 Nanospectralyzer (Applied NanoFluorescence, USA), with a 782 nm excitation laser and a 512 element TE-cooled InGaAs array NIR detector.

In order to quantitatively compare the fluorescence intensities of the nanostar-dye complexes, and thus the fluorescence enhancements of the solid versus core-shell nanostars, the amount of bound dye molecules on the nanostars was quantified. To this end, after addition of the dye to the PEGylated nanostars and incubation overnight, the remaining, unattached dye was removed by centrifugation and collected. Calibration curves of fluorescence against free dye were made and the concentration of unbound dye in the supernatants was determined by measuring the emission at 900 nm and 997 nm for ICG and IR-E1050-COOH respectively.

The amount of dye remaining in the nanostar solutions was calculated by subtraction from the total dye added initially. By knowing the number of nanostars present in solution, an estimate of the dye coverage was also calculated. Using this information, corresponding control solutions of dye in water were prepared to compare against the nanostar-dye complexes.

### Reagents

Gold chloride trihydrate (HAuCl₄·3H₂O), silver nitrate (AgNO₃), sodium citrate tribasic dehydrate, L-ascorbic acid (AA), cetyltrimethylammonium chloride solution (25 wt. %), sodium bromide (NaBr), phosphate buffered saline (PBS), 1-ethyl-3-(3-dimethylaminopropyl)carbodimide hydrochloride (EDC), hydrogen peroxide solution (H₂O₂; 30 wt. %), n-hydroxysuccinimide (NHS), ammonium hydroxide solution (NH₄OH; 30%), sodium borohydride (NaBH₄), 2-(N-morpholino)ethanesulfonic acid (MES) and poly (ethylene glycol) (PEG) methyl ether thiol (2000 Da) were bought from Sigma-Aldrich, United Kingdom. Sulphuric acid (H₂SO₄; 95%), acetone, 2-propanol, ethanol, nitric acid (HNO₃; 68%), hydrochloric acid (37%) were acquired from VWR International, United Kingdom. Carboxylate-functionalised polystyrene spheres (PS) (4% solid; 60 and 80 nm diameter) were purchased from Thermo Fisher Scientific. Thiol-PEG-Amine, HCl salt (MW: 7500) was bought from JenKem Technology, Texas, USA. IR-E1050-COOH was purchased from NIRMidas Biotech, California, USA. Indocyanine green was acquired from Fisher Scientific. Deionized water was purified using the Millipore Milli-Q gradient system (>18.2 Me).

### Example 4

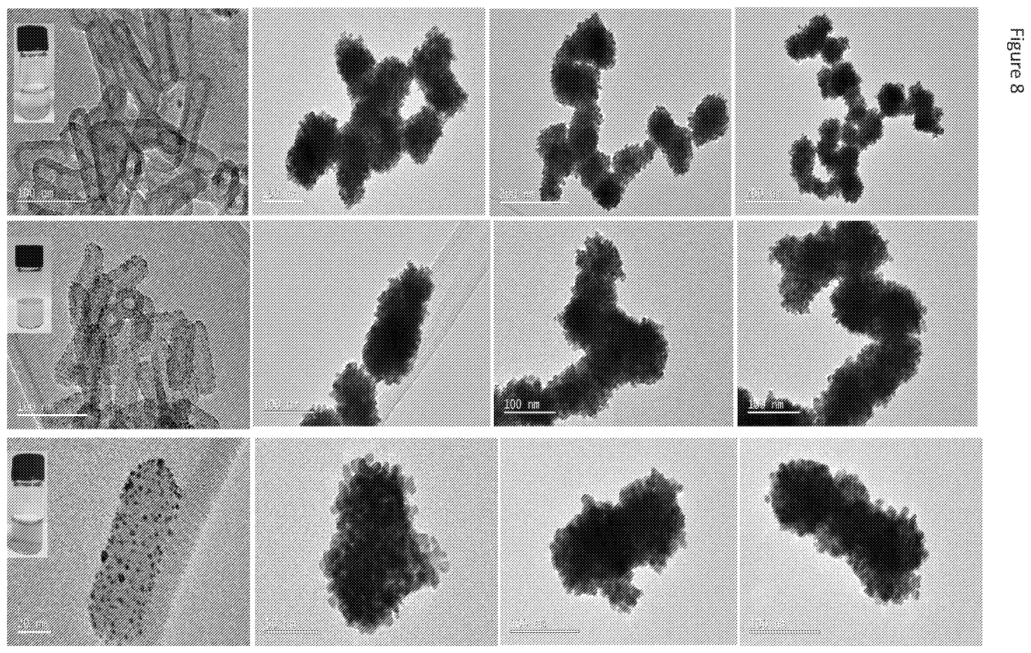

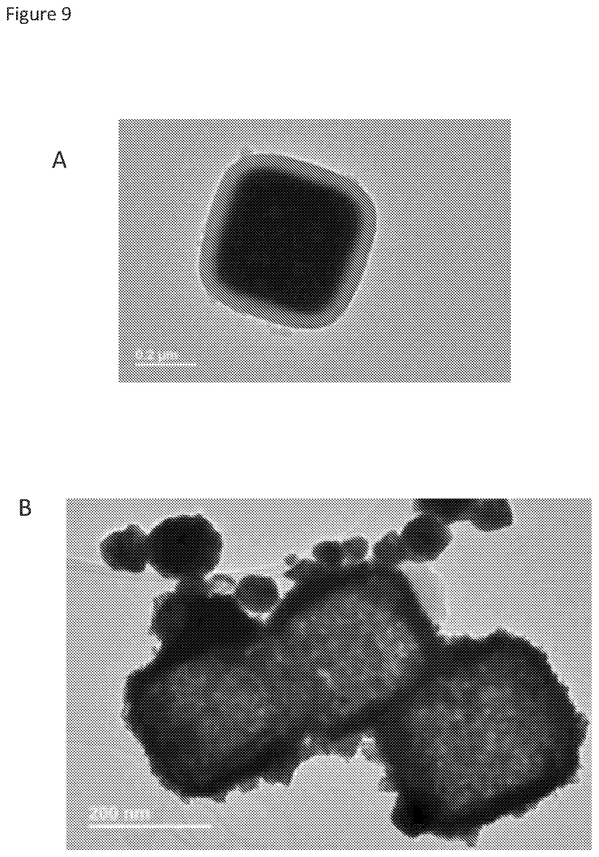

FIGS. 8 and 9 show the nanostructure that take a nanotube and a cuboid shape. These structures were made generally according to the methods of Example 3.

### For FIG. 8 Method:

Briefly, 8.5 g of polyoxyethylene (10) cetyl ether pellet was dissolved in 15 ml of cyclohexane at 55° C. The mixture solution was stirred for 30 min until becoming transparent, followed by adding 1 ml of nickel chloride solution (0.8 M) and 0.45 ml of hydrazine hydrate. The color of the solution quickly turned from faint green to cyan, purple, and pink after 4 h of vigorous stirring, Indicating the formation of nickel-hydrazine nanoparticles. The size of the nickel-hydrazine nanoparticles can be changed by adding different volumes of nickel chloride solution (0.8 M). Later, 30 μl of APTES and 1 ml of diethylamine were simultaneously added into the solution. After 3 h of stirring, 2.3 ml of TEOS was added for silica coating. To keep a constant thickness of the silica shell in each synthesis, the volume of TEOS added was proportional to the nickel chloride. In our synthesis, the volume ratio between TEOS and NiCl2 (0.8 M) was constant with a ratio of 2.3:1. The silica shell growth was complete after 4 h, and the product was then washed twice with isopropanol and redispersed in 25 ml of isopropanol.

Following this, the particles were dispersed in 5 mL ethanol and 15 uL APTES was added, followed by 5 uL NH₄OH (28%). The mixture was stirred overnight. The APTES functionalised hollow silica cubes were then centrifuged and dispersed in ethanol.

For gold seeding, the particles were added to 5 mL of gold ‘Duff solution’ (45 mL water, 0.5 mL THPC, 1 mL NaOH) along with 1 mL NaCl and placed in an orbital shaker for 3 days in the dark. These were cleaned by centrifugation at 3000 rpm and redispersed in water. Full gold shell growth was achieved by adding an aliquot of the seeded cubes into 4 mL of aged K-gold solution (45 mL water, 12.3 mg K2CO3 and 1 mL of 25 mM HAuCl₄·3H₂O) along with 50 μL of 132 mM glucose solution and heated to 80° C. for 5 min. The resulting particles were centrifuged at 3000 rpm for 10 min and redispersed in water.

For spikey star formation, 300 μL of nanoparticles were added to 10 mL of 0.3 mM HAuCl₄·3H₂O, containing 10 μL of 1 M HCl. 150 μL of 2 mM AgNO₃ and 50 μL of 100 mM AA were then quickly added and was stirred for 30 seconds. [44]

### For FIG. 9 Method:

To make 80 nm cuprous oxide cubes, 8.1 mL of deionized water were introduced into 0.0576 g of SDS. After stirring for 5 min to dissolve SDS, 100 μL of 0.1 M CuSO4 solution was added. Next, 80 μL of 1.0 M NaOH were respectively added, followed by the addition of 1.7 mL of 0.2 M sodium ascorbate solution with stirring for 5 min and aging for 10 min. The particles were collected by centrifugation at 9500 rpm for 10 min. Final precipitate was dispersed in 1 mL of ethanol for storage and analysis. [42] Cuprous oxide nanocubes of 300 nm were produced via a seed-mediated synthesis approach by Kuo et al. Briefly, a 140 mL solution containing 10-3 M copper sulfate (CuSO4.5H₂O) and 3.3×10-2 M sodium dodecyl sulfate(SDS) was made using DI water (>18.2 Mg, purified using Millipore Milli-Q gradient system). 9 mL was added into 4 glass vials labelled from B to E and 10 mL added into 1 labelled A. 9 mL was also transferred into 10 glass vials labelled from E1 to E10. 250 μL of a 0.2 M solution of sodium L-ascorbate was added into vial A and stirred for 5 seconds. Following this, 0.5 mL of a 1 M solution of NaOH was added to vial A and stirred for another 5 seconds. Then, a 1 mL aliquot was taken from vial A and added into B. Another 250 μL of sodium L-ascorbate was added and stirred, followed by 0.5 mL of NaOH. 1 mL was then transferred from vial B to C. This process was repeated for all vials from to E(1-10). Cuprous oxide nanocubes were attained after 2 hours. Cubes attained from E vials were centrifuged at 3000 rpm and stored in ethanol. [43]

For silica shell growth, optimised volumes of ethanol, DI water, and tetraorthosilicate (TEOS) were added. For 80 nm cubes, PVP-55 was added as a surfactant and for 200 nm cubes, Triton x-100 was added. The silica mixtures were stirred overnight and then centrifuged and dispersed in water. 1 M HCl was then added dropwise till the particles turned from bright orange to almost colourless, indicating dissolution of the cuprous core. These were centrifuged again and stored in ethanol.

Following this, the particles were dispersed in 5 mL ethanol and 15 uL APTES was added, followed by 5 uL NH₄OH (28%). The mixture was stirred overnight. The APTES functionalised hollow silica cubes were then centrifuged and dispersed in ethanol.

For gold seeding, the particles were added to 5 mL of gold ‘Duff solution’ (45 mL water, 0.5 mL THPC, 1 mL NaOH) along with 1 mL NaCl and placed in an orbital shaker for 3 days in the dark. These were cleaned by centrifugation at 3000 rpm and redispersed in water. Full gold shell growth was achieved by adding an aliquot of the seeded cubes into 4 mL of aged K-gold solution (45 mL water, 12.3 mg K2CO3 and 1 mL of 25 mM HAuCl₄·3H₂O) along with 50 μL of 132 mM glucose solution and heated to 80° C. for 5 min. The resulting particles were centrifuged at 3000 rpm for 10 min and redispersed in water.

For spikey star formation, 300 μL of nanoparticles were added to 10 mL of 0.3 mM HAuCl4·3H₂O, containing 10 μL of 1 M HCl. 150 μL of 2 mM AgNO₃ and 50 μL of 100 mM AA were then quickly added and was stirred for 30 seconds. [44]

### REFERENCES

1. Weissleder, R. A clearer vision for in vivo imaging. Nat. Biotechnol. 19, 316-317 (2001).
2. Diao, S. et al. Biological imaging without autofluorescence in the second near-infrared region. Nano Res. 8, 3027-3034 (2015).
3. Hong, G. et al. Near-Infrared II Fluorescence for Imaging Hindlimb Vessel Regeneration With Dynamic Tissue Perfusion Measurement. Circ. Cardiovasc. Imaging 7, 517-525 (2014).
4. Hong, G. et al. Multifunctional in vivo vascular imaging using near-infrared II fluorescence. Nat. Med. 18, 1841-1846 (2012).
5. Li, B., Lu, L, Zhao, M., Lei, Z. & Zhang, F. An Efficient 1064 nm NIR-II Excitation Fluorescent Molecular Dye for Deep-Tissue High-Resolution Dynamic Bioimaging. Angew. Chemie Int. Ed. 57, 7483-7487 (2018).
6. Hong, G., Antans, A. L & Dai, H. Near-infrared fluorophores for biomedical imaging. Nat. Biomed. Eng. 1, 0010 (2017).
7. Zheng, X., Xing, D., Zhou, F., Wu, B. & Chen, W. R. Indocyanine Green-Containing Nanostructure as Near Infrared Dual-Functional Targeting Probes for Optical Imaging and Photothermal Therapy. Mol. Pharm. 8, 447-456 (2011).
8. Byrd, B. K. et al. Characterizing short-wave infrared fluorescence of conventional near-infrared fluorophores. J. Biomed. Opt. 24, 1 (2019).
9. Geddes, C. D. & Lakowicz, J. R. Editorial Metal-Enhanced Fluorescence concentrating the local field and subsequently increasing nearby metal can increase the intrinsic radiative decay. J. Fluoresc. 12, (2002).
10. Deng, W. & Goldys, E. M. Plasmonic Approach to Enhanced Fluorescence for Applications in Biotechnology and the Life Sciences. Langmuir 28, 10152-10163 (2012).
11. Chen, J., Jin, Y., Fahruddin, N. & Zhao, J. X. Development of Gold Nanoparticle-Enhanced Fluorescent Nanocomposites. Langmuir 29, 1584-1591 (2013).
12. Asian, K., Lakowicz, J. R. & Geddes, C. D. Plasmon light scattering in biology and medicine: new sensing approaches, visions and perspectives. Curr. Opin. Chem. Biol. 9, 538-544 (2005).
13. Theodorou, I. G. et al. Gold Nanostar Substrates for Metal-Enhanced Fluorescence through the First and Second Near-infrared Windows. doi:10.1021/acs.chemmater.7b02313.
14. Guerrero-Martinez, A., Barbosa, S., Pastoriza-Santos, I. & Liz-Marzán, L M. Nanostars shine bright for you: Colloidal synthesis, properties and applications of branched metallic nanoparticles. Curr. Opin. Colloid Interface Sci. 16, 118-127 (2011).
15. Sanchez-Gaytan, B. L. et al. Controlling the Topography and Surface Plasmon Resonance of Gold Nanoshells by a Templated Surfactant-Assisted Seed Growth Method. J. Phys. Chem. C 117, 8916-8923 (2013).
16. Chirico, G., Pallavicini, P. & Borzenkov, M. Physical properties of gold nanostars. Gold Nanostars: Synthesis, Properties and Biomedical Application (2015). doi:10.1007/978-3-319-20768-1_2.
17. Favi, P. M. et al. Shape and surface effects on the cytotoxicity of nanoparticles: Gold nanospheres versus gold nanostars. J. Biomed. Mater. Res.—Part A 103, 3449-3462 (2015).
18. Feng Hao, Colleen L. Nehl, Jason H. Hafner, and & Nordlander*, P. Plasmon Resonances of a Gold Nanostar. (2007) doi:10.1021/NL062969C.
19. Sanchez-Gaytan, B. L. et al. Spiky Gold Nanoshells: Synthesis and Enhanced Scattering Properties. J. Phys. Chem. C 116, 10318-10324 (2012).
20. Lakowicz, J. R. Radiative decay engineering 5: metal-enhanced fluorescence and plasmon emission. Anal. Biochem. 337, 171-194 (2005).
21. Du, B. et al. Hybrid of gold nanostar and indocyanine green for targeted imaging-guided diagnosis and phototherapy using low-density laser irradiation. J. Mater. Chem. B 4, 5842-5849 (2016).
22. Yuan, H. et al. In vivo particle tracking and photothermal ablation using plasmon-resonant gold nanostars. Nanomedicine Nonotechnology, Biol. Med. 8, 1355-1363 (2012).
23. Yuan, H. et at. Gold nanostars: surfactant-free synthesis, 3D modelling, and two-photon photoluminescence imaging. Nanotechnology 23, 075102 (2012).
24. Sanchez-Gaytan, B. L & Park, S. J. Spiky Gold Nanoshells. Langmuir 26, 19170-19174 (2010).
25. Bardhan, R., Grady, N. K., Cole, J. R., Joshi, A. & Halas, N. J. Fluorescence Enhancement by Au Nanostructures: Nanoshells and Nanorods. ACS Nano 3, 744-752 (2009).
26. Catherine J. Murphy, Anand M. Gole, Simona E. Hunyadi, and & Orendorff, C. J. One-Dimensional Colloidal Gold and Silver Nanostructures. (2006) doi:10.1021/IC0519382.
27. Colleen L. Nehl, †, Hongwei Liao, ‡ and & Jason H. Hafner*, †, ‡. Optical Properties of Star-Shaped Gold Nanoparticles. (2006) doi:10.1021/NL052409Y.
28. Rahme, K. et al. PEGylated gold nanoparticles: polymer quantification as a function of PEG lengths and nanoparticle dimensions. RSC Adv. 3, 6085-6094 (2013).
29. Gandra, N. et al. Probing Distance-Dependent Plasmon-Enhanced Near-Infrared Fluorescence Using Polyelectrolyte Multilayers as Dielectric Spacers. Angew. Chemie Int. Ed. 53, 866-870 (2014).
30. Wu, K. et at. Plasmon-enhanced fluorescence of PbS quantum dots for remote near-infrared imaging. Chem. Commun. 51, 141-144 (2015).
31. Jana, J., Aditya, T., Ganguly, M., Mehetor, S. K. & Pal, T. Fluorescence enhancement via varied long-chain thiol stabilized gold nanoparticles: A study of far-field effect. Spectrochim. Acta Part A Mol. Biomol. Spectrosc. 188, 551-560 (2018).
32. Geddes, C. D. et al. Metal-enhanced fluorescence (MEF) due to silver colloids on a planar surface: Potential applications of indocyanine green to in vivo imaging. J. Phys. Chem. A 107, 3443-3449 (2003).
33. Bardhan, R., Grady, N. K. & Halas, N. J. Nanoscale control of near-infrared fluorescence enhancement using au nanoshells. Small 4, 1716-1722 (2008).
34. Starosolski, Z. et at. Indocyanine green fluorescence in second near-infrared (NIR-II) window. PLoS One 12, (2017).
35. Bhavane, R., Starosolski, Z., Stupin, I., Ghaghada, K. B. & Annapragada, A. NIR-II fluorescence imaging using indocyanine green nanoparticles. Sci. Rep. 8, 14455 (2018).
36. Cai, Z. et al. NIR-II fluorescence microscopic imaging of cortical vasculature in non-human primates. Theranostics 10, 4265-4276 (2020).
37. Fang, S. et al. Gold Nanorods-Based Theranostics for Simultaneous Fluorescence/Two-Photon Luminescence Imaging and Synergistic Phototherapies. J. Nanomater. 2016, (2016).
38. Kinkhabwala, A. et a). Large single-molecule fluorescence enhancements produced by a bowtie nanoantenna. Nat. Photonics 3, 654-657 (2009).
39. Pallavicini, P. et al. Gold nanostars coated with neutral and charged polyethylene glycols: A comparative study of in-vitro biocompatibility and of their interaction with SH-SY5Y neuroblastoma cells. 151, 123-131 (2015).
40. Chen, H. et al. Multifunctional gold nanostar conjugates for tumor imaging and combined photothermal and chemo-therapy. Theranostics 3, (2013).
41. Espinosa, A. et al. Cancer Cell Internalization of Gold Nanostars Impacts Their Photothermal Efficiency in Vitro and in Vivo: Toward a Plasmonic Thermal Fingerprint in Tumoral Environment. Adv. Healthc. Mater. 5, 1040-1048 (2016).
42. Scalable Synthesis of Size-Tunable Small Cu₂O Nanocubes and Octahedra for Facet-Dependent Optical Characterization and Pseudomorphic Conversion to Cu Nanocrystals Subashchandrabose Thoka, An-Ting Lee, and Michael H. Huang ACS Sustainable Chemistry & Engineering 2019 7 (12), 10467-10476 DOI: 10.1021/acssuschemeng.9b00844
43. Kuo, C. H., Chen, C. H. and Huang, M. (2007), Seed-Mediated Synthesis of Monodispersed Cu₂O Nanocubes with Five Different Size Ranges from 40 to 420 nm. Adv. Funct. Mater., 17: 3773-3780. https://doi.org/10.1002/adfm.200700356
44. Nanoscale, 2018,10, 15854-15864

1. A nanostructure, wherein the nanostructure comprises:
a) a dielectric core material or a hollow core;
b) a metallic plasmonic material; and
c) an emitter that absorbs and/or emits electromagnetic radiation in the near infra-red II window (NIR-II) and/or in the near infra-red III (NIR-III) window;
wherein the metallic plasmonic material covers substantially all, or covers all, of the dielectric core and optionally wherein the nanostructure comprises at least one spike.

2. The nanostructure according to claim 1 wherein:
the NIR-II window is defined as electromagnetic radiation of a wavelength of between 1,000 nm to 1,400 nm; and
the NIR-III window is defined as electromagnetic radiation of a wavelength of: 1400 nm to 1700 nm.

3. The nanostructure according to either of claims 1 or 2, wherein the emitter that absorbs and/or emits electromagnetic radiation in the near infra-red II window (NIR-II) and/or in the near infra-red III (NIR-III) window is selected from:
a) a fluorescent dye, optionally an organic dye, optionally Indocyanine green (ICG) or IR-E1050;
b) Inorganic emitters, optionally a quantum dot, optionally Ag₂S QDs; and/or
c) a downconversion nanoparticle (DCNP).

4. The nanostructure according to any of claims 1-3 wherein the nanostructure also comprises a seed structure, wherein the seed structure is present on the surface of the dielectric core, and is also covered by the metallic plasmonic material, optionally wherein the seed structure comprises a metallic material that is different from the plasmonic material that coats the dielectric core.

5. The nanostructure according to any of claims 1-4, wherein a cross section of the core substantially describes a circle, a square, triangle or a rectangle.

6. The nanostructure according to any of claims 1-5, wherein the shape of the core is substantially spherical, substantially hexahedral, substantially cuboid, substantially rectangular cuboid, triangular prism, pyramidal, substantially cylindrical, substantially tubular or substantially rod-like.

7. The nanostructure according to any of claims 1-6, wherein the dielectric material is:
a solid, optionally is a polymer, optionally wherein the polymer is selected from the group comprising or consisting: polystyrene, silica or any combination thereof, optionally wherein the dielectric core is polystyrene; or
is a liquid;
is a gas, optionally is dry air, Ammonia, Air, Carbon dioxide, Sulphur hexafluoride (SF6), Carbon Monoxide, Nitrogen, Hydrogen.

8. The nanostructure according to any of claims 1-7, wherein the metallic plasmonic material is selected from the group comprising or consisting:
a noble metal or a salt thereof;
a base metal or a salt thereof;
gold or a salt thereof;
silver or a salt thereof;
copper or a salt thereof;
aluminium or a salt thereof;
or any combination thereof.

9. The nanostructure according to any of claims 1-8, wherein the metallic plasmonic material is:
a) gold;
b) silver;
c) copper;
d) aluminium; or
e) an alloy of any one or more of gold, silver, copper and/or aluminium.

10. The nanostructure according to any of claims 1-9, wherein the average diameter (a) of the metallic material-coated nanostructure is:
from 1 nm to 1000 nm, optionally from 50 nm to 900 nm; 100 nm to 800 nm; 200 nm to 700 nm; 300 nm to 600 nm, 400 to 500 nm; and/or
at least inm, 50 nm, 100 nm, 200 nm, 300 nm, 400 nm, 500 nm, 600 nm, 700 nm, 800 nm, 900 nm and 1000 nm; and/or
less than 1000 nm, 900 nm, 800 nm, 700 nm, 600 nm, 500 nm, 400 m, 300 nm, 200 nm, 100 nm, 50 nm, 25 nm, or 5 nm, or 1 nm.

11. The nanostructure according to any of the preceding claims, wherein the average diameter (b) of the dielectric core or hollow core is:
from 1 nm to 1000 nm, optionally from 50 nm to 900 nm; 100 nm to 800 nm; 200 nm to 700 nm; 300 nm to 600 nm, 400 to 500 nm; and/or
at least 1 nm, 50 nm, 100 nm, 200 nm, 300 nm, 400 nm, 500 nm, 600 nm, 700 nm, 800 nm, 900 nm and 1000 nm;
less than 1000 nm, 900 nm, 800 nm, 700 nm, 600 nm, 500 nm, 400 m, 300 nm, 200 nm, 100 nm, 50 nm, 25 nm, or 5 nm, or 1 nm; and/or
or is 60 nm or 80 nm.

12. The nanostructure according to any of the preceding claims, wherein the metal coating (c) has a maximum average thickness of:
at least 1 nm, 2, 3, 4, 5, 6, 7, 8, 9, 10, 11, 12, 13, 14, 15, 20, 30, 40, 50, 60, 70, 80, 90, 100, 150, 200, 250, 300, 350, 400, 450, 500, 550, 600, 650, 700, 750, 800, 850, 900, 950 or at least 1000 nm; and/or
less than 1000 nm, 950, 900, 850, 800, 750, 700, 650, 600, 550, 500, 450, 400, 350, 300, 250, 200, 150, 100, 90, 80, 70, 60, 50, 40, 30, 20, 15, 14, 13, 12, 11, 10, 9, 8, 7, 6, 5, 4, 3, 2 or 1 nm; and/or
from 1 nm to 1000 nm, 2 to 950, 3 to 900, 4 to 850, 5 to 800, 6 to 750, 7 to 700, 8 to 650, 9 to 600, 10 to 550, 11 to 500, 12 to 450, 13 to 400, 14 to 350, 15 to 300, 20 to 250, 30 to 200, 40 to 150, 50 to 100, 60 to 90, 70 to 80 nm.

13. The nanostructure according to claim 12, wherein the nanostructure is star-shaped or substantially star-shaped.

14. The nanostructure according to claim 13, wherein the coating comprises a plurality of spikes extending from the surface of the metallic coating.

15. The nanostructure according to claim 14, wherein:
a) the surface of the spikes is contiguous with the surface of the metallic coating; and/or
b) the spikes constitute part of the metallic coating.

16. The nanostructure according to any of claims 14 or 15, wherein the spikes comprise or consist the same metal plasmonic material as the coating.

17. The nanostructure according to any of claims 14-16, wherein:
A) the average length (d) of the spikes is:
i) between 20 to 60 nm; for example between 25 nm and 55 nm, 30 nm and 50 nm, 35 nm and 45 nm;
ii) of at least 20 nm, for example at least 20, 25, 30, 35, 40, 45, 50, 55, or at least 60 nm; and/or
iii) of less than 60 nm, for example less than 60 nm, 55, 50, 45, 40, 35, 30, 25, 20 nm;
B) the average diameter of the base of the spikes is:
i) between 20 to 60 nm, for example between 25 to 55, 30 to 50, 35 to 45 or 40 nm;
ii) at least 20 nm, for example at least 25, 30, 35, 40, 45, 50, 55, 60 nm; and/or
iii) less than 60 nm, for example less than 55, 50, 45, 40, 35, 30, 25 or less than 20 nm;
C) the average diameter of the tip of the spike is:
i) between 4 nm to 38 nm, for example between 6 to 36, 8 to 34, 10 to 32, 12 to 30, 14 to 28, 16 to 26, 18 to 24, 20 to 22 nm;
ii) at least 4 nm, for example at least, 6, 8, 10, 12, 14, 16, 18, 20, 22, 24, 26, 28, 30, 32, 34, 36, or at least 38 nm;
iii) less than 38 nm, for example less than 36, 34, 32, 30, 28, 26, 24, 22, 20, 18, 16, 14, 12, 10, 8, 6, or less than 4 nm; and/or
D) the average aspect ratio of the spikes is:
i) greater than 1.2;
ii) 1
iii) less than 1.

18. The nanostructure according to any of the preceding claims, further comprising a target binding agent capable of binding specifically to a target;
optionally wherein the target binding agent is:
a protein, optionally wherein the protein is selected from the group comprising or consisting: an antibody or antigen binding fragment thereof; or
a nucleic acid, optionally an aptamer.

19. The nanostructure according to claim 18, wherein the target is a protein, optionally:
a) a tumour associated antigen (TAA), optionally selected from the group comprising or consisting:
alpha-actinin-4 (ACTN4; optionally UniProtKB—043707); AP2S1 (optionally UniProtKB—P53680); BBX (optionally UniProtKB—Q8WY36); BRAF (optionally UniProtKB—P15056); BCR-ABL fusion; CTNNB1 (optionally UniProtKB—P35222); CASP5 (optionally UniProtKB—P51878); CASP8 (optionally UniProtKB—Q14790); CDC27 (optionally UniProtKB—P30260); CDK12 (optionally UniProtKB—Q9NYV4); CDK4 (optionally UniProtKB—P11802); CDKN2A (optionally UniProtKB—P42771); CLPP (optionally UniProtKB—Q16740); UBXN11 (optionally UniProtKB—Q5T124); CSNK1A1 (optionally UniProtKB—P48729); dek-can fusion protein; EFTUD2 (optionally UniProtKB—Q15029); EEF2 (optionally UniProtKB—P13639); ETV6-AML1 fusion protein; FLT3 (optionally UniProtKB—P36888); FN1 (optionally UniProtKB—P02751); FNDC3B (optionally UniProtKB—Q53EP0); GAS7 (optionally UniProtKB—060861); GPNMB (optionally UniProtKB—Q14956); HAUS3 (optionally UniProtKB—Q68CZ6); HLA-A (optionally UniProtKB—P04439); HSDL1 (optionally UniProtKB—Q3SXM5); HSPA2 (optionally UniProtKB—P54652); KRAS (optionally UniProtKB—P01116); KIAAO205; LDLR-fucosyltransferaseAS fusion protein; HHAT (optionally UniProtKB—Q5VTY9); MATN1 (optionally UniProtKB—P21941); ME1 (optionally UniProtKB—P48163); TRAPPC1 (optionally UniProtKB Q9Y5R8); MUM-3; MYO1B (optionally UniProtKB—043795); NRAS (optionally UniProtKB—P01111); PAPOLG (optionally UniProtKB—Q9BWT3); NFYC (optionally UniProtKB—Q13952); OGT (optionally UniProtKB—015294); OS9 (optionally UniProtKB—Q13438); TP53 (optionally UniProtKB—P04637); pml-RARalpha fusion protein; PPP1R3B (optionally UniProtKB—Q86XI6); PRDX5 (optionally UniProtKB—P30044); PTPRK (optionally UniProtKB—Q15262); UBR4 (optionally UniProtKB—Q5T4S7); SIRT2 (optionally UniProtKB—Q8IXJ6); SNRPD1 (optionally UniProtKB—P62314); SYT-SSX1 or —SSX2 fusion protein; TGFBR2 (optionally UniProtKB—P37173); TPI1 (optionally UniProtKB—P60174); (optionally UniProtKB—);
b) a tumour neoantigen;
c) a target associated with a pathogen, optionally associated with a bacterial cell, a virus or a parasite; and/or
d) a target associated with an analyte in a sample obtained from a subject.

20. The nanostructure according to any of the preceding claims, wherein the emitter is bound to the nanostructure by a linker, optionally wherein the linker is a polyethylene glycol (PEG) linker, PVP or a carbodiimide linker

21. A method of:
a) metal enhanced fluorescence;
b) plasmon enhanced fluorescence; and/or
c) enhancing the fluorescence signal emitted by an emitter, optionally a fluorophore
comprising using the nanostructure according to any of the preceding claims.

22. A composition comprising:
a) the nanostructure according to any of claims 1-20; or
b) at least a first plurality of nanostructures according to any of claims 1-20 and at least a second plurality of nanostructures according to any of claims 1-20, wherein the nanostructures of the first plurality are different to the nanostructures of the second plurality.

23. A pharmaceutical composition comprising the nanostructure according to any of claims 1-20.

24. The nanostructure according to any of claims 1-20, or the composition according to claim 22, or the pharmaceutical composition according to claim 23 for use in medicine.

25. A method for enhanced resolution of in situ fluorescence imaging of biological tissue wherein the method comprises the use of the nanostructure according to any of claims 1-20 or the composition according to claim 22 or the pharmaceutical composition according to claim 23,
optionally wherein the method is a method for enhanced imaging of a tumour.

26. An in vitro method of determining the presence or level of a component in a sample, wherein the method comprises:
contacting a plurality of the nanostructures according to any of claims 1-20 with the sample, wherein the nanostructures comprise a target binding agent and wherein said contacting allows binding of the target binding agent to the target if present in the sample,
optionally wherein the sample is a sample obtained from a subject, optionally wherein the sample is selected from the group comprising or consisting of: blood, plasma, mucus, transudate, urine, milk, phlegm, saliva, bile, semen, tears, pus, sebum, intracellular fluid, interstitial fluid, cerebrospinal fluid, lymphatic fluid, tissue sample, bone sample, bone marrow sample, breast sample, gastrointestinal sample, lung sample, liver sample, pancreatic sample, prostate sample, brain sample, nerve sample, meningeal sample, renal sample, endometrial sample, cervical sample, lymph sample, muscle sample, skin sample, or any combination thereof.

27. An in vitro diagnostic method comprising the method of claim 26.

28. A method of producing a nanostructure, wherein the nanostructure is the nanostructure according to any of claims 1-20, optionally wherein
the method comprises:
a) providing at least one dielectric core;
b) decorating the at least one dielectric core with a plurality of seeds; and
c) incubating the decorated dielectric core in a growth solution,
optionally wherein
a) Incubation of the decorated dielectric core in the growth solution coats the decorated dielectric core in a plasmonic metallic coating;
b) the dielectric core is composed of a polymer, optionally wherein the polymer is selected from the group comprising or consisting of: polystyrene, silica or any combination thereof, optionally wherein the dielectric core is polystyrene;
c) the seeds are composed of metal, optionally wherein the metal is selected from the group comprising or consisting of: a noble metal or a salt thereof; a base metal or a salt thereof; gold or a salt thereof; silver or a salt thereof; copper or a salt thereof; aluminium or a salt thereof; or any allow thereof, or any combination thereof;
d) the growth solution comprises a metal, optionally wherein the metal is selected from the group comprising or consisting of: a noble metal or a salt thereof; a base metal or a salt thereof; gold or a salt thereof; silver or a salt thereof; copper or a salt thereof; aluminium or a salt thereof; or any combination thereof; and/or
e) the growth solution comprises an anionic surfactant, optionally comprises any one or more of CTAC or CTAB, and optionally further comprises NaBr.

29. A method for imaging comprising the use of the nanostructure according to any of claims 1-20 or the composition according to claim 22 or the pharmaceutical composition according to claim 23,
optionally wherein the method is a method for imaging of a tumour.

30. A method for combined imaging and photothermal therapy comprising the use of the nanostructure according to any of claims 1-20 or the composition according to claim 22 or the pharmaceutical composition according to claim 23,
optionally wherein the method is a method for imaging of a tumour.
